(19) United States
(12) Patent Application Publication
(10) Pub. No.: US 2026/0258352 A1
(43) Pub. Date: Sep. 3, 2026

(54) INDUCED PHOTORECEPTOR CELLS AND METHODS FOR THEIR PRODUCTION

(71) Applicant: Rheinische Friedrich-Wilhelms-Universitat Bonn, Bonn (DE)

(72) Inventors: Volker Busskamp, Bonn (DE); Marta Zuzic, Bonn (DE); Anka Kempe, Dresden (DE)

(73) Assignee: Rheinische Friedrich-Wilhelms-Universitat Bonn, Bonn (DE)

(21) Appl. No.: 19/361,212

(22) Filed: Oct. 17, 2025

Related U.S. Application Data
(63) Continuation of application No. 17/435,235, filed on Aug. 31, 2021, now Pat. No. 12,448,602.
Application No. 17/435,235 is a continuation-in-part of application No. PCT/EP2020/055401, filed on Mar. 2, 2020.

(30) Foreign Application Priority Data
Mar. 4, 2019 (EP) 19160600.3
Apr. 23, 2019 (EP) 19170478.2

(51) Int. Cl. C12N 5/0793; A61K 35/30; A61P 27/02

(52) U.S. Cl. CPC C12N 5/062; A61K 35/30; A61P 27/02

(57) ABSTRACT

A method for producing induced photoreceptor cells from an initial cell is provided, the method includes providing one or more transcription factors (TFs) including at least GON4L to the initial cell. In some versions, the initial cell is a human induced pluripotent stem cell (iPSC). In other embodiments the method includes providing the TFs OTX2 and/or NEUROD1 to the initial cell. Cells produced and obtainable by the method are also provided, the use of these cells as a medicament in the treatment of retinopathy, vectors for inducing the photoreceptor cells and combinations of transcription factors intended for this use.

### RELATED APPLICATIONS

This application is a continuation of U.S. application Ser. No. 17/435,235, filed Aug. 31, 2021; that in turn is a US National Phase filing of International Application Serial Number PCT/EP2020/055401, filed Mar. 2, 2020; that in turn claims priority benefit of EP Application Serial Numbers 19160600.3 and 19170478.2 with filing dates of Mar. 4, 2019 and Apr. 23, 2019; respectively; the contents of which are hereby incorporated by reference.

### SEQUENCE LISTING

A sequence listing containing the nucleotide and/or amino acid sequence information that is in an electronic format (XML) is filed via the USPTO patent electronic filing system is incorporated herein by reference. The electronic ST26 format file created on Apr. 2, 2026 and being 35,000 bytes in size and entitled “HERT-0137USCON”. The sequence listing is located in the electronic file associated with this application. The specification is amended to include an incorporation by reference statement in compliance with 37 CFR 1.821(c) and 1.831-1.834.

### FIELD OF THE INVENTION

The invention relates to a method for producing induced photoreceptor cells from an initial cell, the method comprising providing one or more transcription factors (TFs) comprising at least GON4L to the initial cell. In preferred embodiments of the invention, the initial cell is a human induced pluripotent stem cell (iPSC). In other embodiments the method comprises providing the TFs OTX2 and/or NEUROD1 to the initial cell. The invention further relates to the cells produced and obtainable by the method of the invention, the use of these cells as a medicament in the treatment of retinopathy, vectors for inducing the photoreceptor cells of the present invention and combinations of transcription factors intended for this use.

### BACKGROUND OF THE INVENTION

The use of pluripotent stem cells in regenerative therapy for the treatment of retinal diseases has been discussed in the literature and several approaches for achieving this goal have been suggested (Oswald and Baranov, 2018 “Regenerative medicine in the retina: from stem cells to cell replacement therapy”, Ther Adv Ophthalmol.; Weed and Mills, 2017 “Strategies for retinal cell generation from human pluripotent stem cells”, Stern Cell Investig.). Different methods for the production of photoreceptor cells have emerged. One method promotes the differentiation of photoreceptors from human embryonic stem cells by the addition of growth factors, inhibitors or low-molecular compounds (Zhou et al., 2015 “Differentiation of human embryonic stem cells into cone photoreceptors through simultaneous inhibition of BMP, TGF β and Wnt signaling” Development 2015 Oct. 1; 142(19):3294-306). In another approach, retinal progenitors such as photoreceptor precursor cells are differentiated from mouse iPSCs (Xie et al. PLOS ONE, vol. 9, no. 11, 2014 Nov. 17, page e112175) using manipulation of the Wnt and TGF-beta/BMP signaling pathways by using specific inhibitory molecules.

Furthermore, direct cell conversion from somatic cells (1, 2) or stem cells (via 3D organoids) (3-6) has been suggested. Direct conversion from somatic cells uses transcription factor (TF) overexpression in human fibroblasts and yields photoreceptor-like cells in extremely low quantity. For example, Seko et al. (GENES TO CELLS, vol. 19, no. 3, 2014 Jan. 24, pages 198-208) have derived human photoreceptor cells from fibroblasts by defined combinations of the TF CRX, RAX, OTX2 and NEURD. An alternative approach is to generate human retinal organoids out of human iPSCs that will be dissociated after >100 days in culture, resulting in about 10% photoreceptors that need to be extensively purified.

Photoreceptors need to be enriched from 2D (direct conversion from fibroblasts) or 3D organoids, which is technically challenging as all dissociation and purification protocols are stressful for the cells and depend on specific markers for fluorescence-activated-cell-sorting (FACS) or magnetic-activated-cell-sorting (MACS) (7). Furthermore, human fibroblasts proliferation time is longer compared to human iPSCs, which is important for the amount of starting cell population. 3D retinal organoids need to be cultured for >100 days before photoreceptors can be harvested, which easily results in batch effects reducing the final quality. Longer incubation times and complicated down-stream processing further increase the costs of a medical product for cell transplantation.

Due to extensive studies of in vivo retinogenesis, many TFs important for photoreceptor development are known and applied to human fibroblasts; however, they are insufficient to drive photoreceptor differentiation from human iPSCs or other pluripotent cells, as human iPSCs and photoreceptor progenitor cells differ in their cellular ground state and the knowledge from fibroblast transdifferentation protocols cannot be applied to other initial cells, especially not iPSC.

In order to transplant human photoreceptors into patient retinas for treating blindness diseases, one needs an efficient protocol to derive human photoreceptors in high quantity and quality from human induced pluripotent stem cells (iPSCs). Therefore, a fast, efficient, easy-to-adapt, homogeneous and controllable differentiation protocol needs to be developed to provide human photoreceptors in cell therapy quality.

In light of the prior art there remains a significant need in the art for a fast, efficient and homogeneous differentiation protocol for generating induced photoreceptors from initial cells, such as human iPSC, that provides the cellular quantity and quality of induced photoreceptors for cell transplantation therapies to replace damaged or degenerated photoreceptors.

### SUMMARY OF THE INVENTION

In light of the prior art the technical problem underlying the present invention is to provide alternative or improved methods for producing induced photoreceptor cells. Another object of the invention is the provision of alternative or improved therapeutic agents for treating medical conditions associated with damaged or degenerated photoreceptors. In addressing these objectives, the present invention seeks to avoid the disadvantages of the prior art.

This problem is solved by the features of the independent claims. Preferred embodiments of the present invention are provided by the dependent claims.

The invention therefore relates to a method for producing induced photoreceptor cells from an initial cell, the method comprising providing one or more transcription factors (TFs) comprising at least GON4L to the initial cell.

It was entirely surprising that the transcription factor GON4L, which has never been described in the context of photoreceptor differentiation, is an effective factor for induction of a photoreceptor phenotype in an initial cell to be reprogrammed into a photoreceptor-like cell. It was only possible to identify this completely unexpected TF by performing an unbiased library screening comprising the practically all human TFs. Surprisingly, it was not sufficient to use TFs that were already known to be involved in photoreceptor development to induce differentiation of an initial cell into a photoreceptor-like cell or photoreceptor progenitor cell, but GON4L was required to achieve this.

Importantly, the method of the invention enables fast and efficient induction of a photoreceptor phenotype in the initial cells resulting in a relatively homogenous cellular population, which can be optionally further purified by isolating the induced photoreceptor cells.

In contrast to known 2D cell culture protocols for generating photoreceptor cells from an initial cell, the method of the invention is fast and can be applied to different cell types, including proliferating cells such as iPSC. Provision of the TF GON4L and potentially further TF can occur in a step-wise manner. For example, it is possible to deliver one or more exogenous nucleic acids encoding the required transcription factors to the initial cell without inducing expression of the factors from the exogenous nucleic acid. Subsequently, the initial cells can be expanded for several rounds of replication before inducing expression of the factors from the nucleic acid, which corresponds to the provision of the TFs, so that the initial cells can be massively expanded before inducing photoreceptor differentiation, enabling the generation of large amounts of induced photoreceptor cells form only few initial cells. This represents an important advantage over known 2D differentiation protocols using for example for slowly dividing fibroblasts as an initial cell.

Furthermore, cells displaying a phenotype resembling photoreceptor precursors can be identified in the culture systems very early on after provision of GON4L and potentially further transcription factors. Such early precursor cells as well as cells corresponding more differentiated or mature photoreceptor development stage can be easily isolated by means described herein and known in the art for downstream applications of the cells.

The provision of GON4L, potentially in combination with other TFs, in particular OTX2 and NEUROD1, and/or other factors, represents a novel method for inducing a photoreceptor phenotype in a starting cell in culture.

Without being limited by theory, the use of GON4L for inducing a photoreceptor phenotype is considered necessary to prime the initial cells for photoreceptor differentiation.

It was entirely surprising that provision of GON4L is sufficient for inducing a photoreceptor phenotype in an initial cell, in particular when using iPSC as an initial cell. As is evident form the enclosed examples, GON4L is the only TF required for inducing expression of reporter genes under the control of photoreceptor-specific promoters, although the combined expression of GON4L with other TFs is preferred.

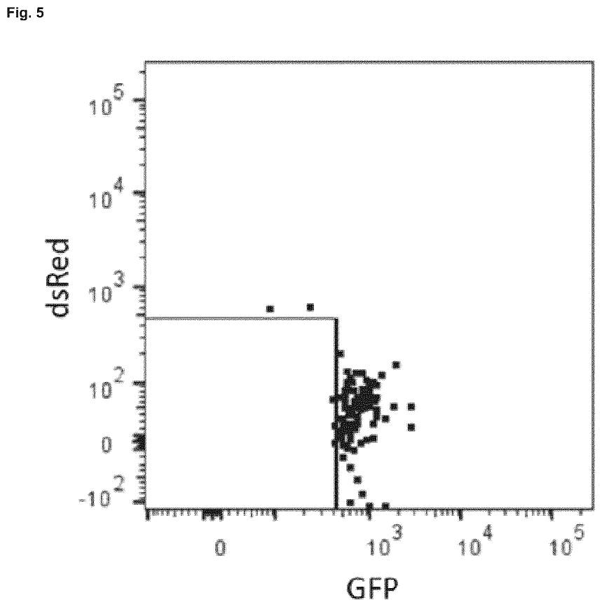

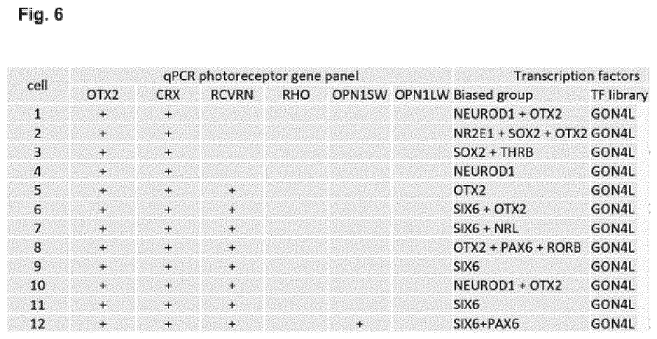

According to the present invention, GON4L expression appears necessary for achieving the technical effect of inducing photoreceptor cells from an initial cell, preferably combined with the expression of another TF for inducing a photoreceptor phenotype, more preferably via the combined expression of GON4L, with OTX2 and/or NEUROD1 (see FIGS. 5 and 6).

A major advantage of the method of the invention is that induced photoreceptor cells can be produced in high purity, which simplifies further downstream processing for purification and enrichment of the cells to a homogenous population.

In embodiments of the invention, the initial cell is a pluripotent or multipotent mammalian cell that is differentiated to the induced photoreceptor cells via providing the one or more transcription factors (TFs) comprising at least GON4L to the initial cell.

Preferably, the initial cell is an induced pluripotent stem cell (iPSC).

It is particularly advantageous to use iPSC as an initial cell for the method of the invention since these cells can be easily expanded due to their proliferative capacity. Accordingly, in embodiments where one or more TFs are provided through expression from one or more nucleic acids in an inducible fashion, it is possible to expand the iPSC after delivery of the nucleic acid, but before induction of TF expression from the nucleic acid. Therefore, it is possible to induce a high number of photoreceptor cells from only a few initial cells. This advantage holds true also for other proliferating or expandable cells that may serve as an initial cell. Furthermore, it is possible to generate iPSC from an individual patient as initial cells for the method of the present invention. Such personal cells can be used as a medicament in the treatment of the same patient after induction of the photoreceptor phenotype by means of the present invention. Accordingly, it is possible to generate patient specific induced photoreceptor cells in high quantities from only a few isolated patient specific cells.

In preferred embodiments of the invention the initial cell is of human origin.

The human origin of the initial cell is particularly advantages since the induced photoreceptor cells generated from such cells will also be human, which is preferable for therapeutic and research applications of the photoreceptor cells of the invention. If the induced photoreceptors are of human origin they can be used for transplantation into patients in need of such cells, for example patients suffering from retinal degeneration or other eye diseases. Furthermore, for the use of the cells of the invention for research and development purposes, for example in drug screening and development, it is a great advantage to use human cells.

In certain embodiments, the initial cell is a fibroblast. Fibroblasts are advantageous initial cells since they are easily accessible from a donor and are easy to culture. Accordingly, it may be possible to generate a high number of fibroblasts from a patient that can be immediately applied as initial cells in the method of the invention leading to fast generation of induced photoreceptor cells after isolation of the cells from the patient. Further preferred initial cells can be bone marrow derived cells, such as hematopoietic stem cells, proliferating precursor cells present in the bone marrow, leukocytes, lymphocytes.

In further embodiments, other somatic or precursor cells may be used as an initial cell. A skilled person is able to select suitable initial cells in view of general knowledge and the state of the art. It has been described that specific cellular phenotypes of differentiated cells can be induced either from stem cells, such as iPSC, or from other initial cells, such as fibroblasts or other somatic cells that may be fully differentiated or still have a differentiation potential. From such studies, a skilled person can conclude that TF that can promote induction of a certain phenotype in a iPSC as an initial cell are also useful for inducing such a specific phenotype in a different cell type used as an initial cell.

The induction of human neuronal cells by defined transcription factor expression has been described previously, whereby somatic cell nuclear transfer, cell fusion, or expression of lineage-specific factors have been shown to induce cell-fate changes in diverse somatic cell types (Pang et al, Nature. 2011 May 26; 476(7359):220-3). For example, forced expression of a different combination of three transcription factors (Brn2, Ascii and Myth I) can efficiently convert mouse fibroblasts and pluripotent cells into functional induced neuronal (iN) cells.

Accordingly, the use of iPSC as an initial cell type in the context of the present invention is a preferred embodiment, but a skilled person would not expect that the use of iPSC as an initial cell is an essential feature of the invention. On the contrary, the fact that GON4L can induce a photoreceptor cell phenotype in iPSC indicates that GON4L can also promote the induction of a photoreceptor phenotype in other initial cell types.

In one embodiment, the initial cell is not an embryonic stem cell or other cell obtained from an embryo.

In embodiments of the invention the induced photoreceptor cell is a cone.

In further embodiments the induced photoreceptor cell is a rod.

In some embodiments, the induced photoreceptor cell is a photosensitive retinal ganglion cell.

It is a great advantage of the method of the invention that by modifying the culture condition of provision of factor combination it is possible to enable directed generation of rods, cones or photosensitive retinal ganglion cells from the initial cells. This is particularly advantageous for using the induced photoreceptor cells in downstream applications that are specific to a certain photoreceptor subtype.

In embodiments of the invention, the method comprises providing one or more TFs selected from CRX, NEUROD1, NR2E1, NR2E3, NRL1, OTX2, ONECUT1, PAX6, RAX, RORB, RXRG, SIX3, SIX6, SOX2, THRB and VSX2 to the initial cell.

In embodiments of the invention, two or more TF comprising GON4L are provided to an initial cell. In another embodiment, three or more TF comprising GON4L are provided to an initial cell. In embodiments of the invention, two or more TF comprising GON4L and one or more TFs selected from the group consisting of CRX, NEUROD1, NR2E1, NR2E3, NRL1, OTX2, ONECUT1, PAX6, RAX, RORB, RXRG, SIX3, SIX6, SOX2, THRB and VSX2 are provided to the initial cell.

It was entirely surprising that the provision of GON4L together with at least another, preferably two additional TF was effective in inducing a photoreceptor phenotype in an initial cell, such as an iPSC. The enclosed examples demonstrate that GON4L in combination with SIX6, NEUROD1 or OTX2, obtained the desired effect. In one embodiment, the invention comprises providing GON4L and OTX2 to an initial cell, such as preferably an iPSC. In one embodiment, the invention comprises providing GON4L and SIX6 to an initial cell, such as preferably an iPSC. In one embodiment, the invention comprises providing GON4L and NEUROD1 to an initial cell, such as preferably an iPSC.

These TFs have been identified to facilitate photoreceptor development from an initial cell when provided in combination with GON4L and to more efficiently induce a photoreceptor phenotype in the initial cell.

In preferred embodiments, the method of the invention comprises providing the TFs OTX2 and/or NEUROD1 to the initial cell.

Surprisingly, it was found out that expression of either OTX2 or NEUROD1, and in particular both TFs, improved the differentiation capacity of an initial cell to an induced photoreceptor when provided in combination with GON4L.

In one embodiment, the combination GON4L, OTX2 and NEUROD1 is provided to the initial cell. In one embodiment, the invention comprises providing GON4L, OTX2 and NEUROD1 to an iPSC as the initial cell. In some embodiments, TFs are provided at about the same time for induction of a photoreceptor phenotype in the initial cell in the context of the method of the invention. In further embodiments, the TFs may be provided sequentially. For example, GON4L may be provided several minutes, hours or days before a second TF, such as OTX2 and/or NEUROD1. A third TF may be provided at the same time as the first or second TF or at a later time point. In embodiments of the invention GON4L is provided after at least one other TF, such as OTX2 and/or NEUROD1. This sequential provision also holds true for further TFs or other factors such as micro-RNAs that may be provided to the initial cell in the context of the method of the invention.

In embodiments of the invention OTX2, NEUROD1 and GON4L are provided to the initial cell at essentially the same time or sequentially.

The order of provision can be (i) GON4L, (ii) OTX2 and (iii) NEUROD1 or (i) GON4L, (ii) NEUROD1 and (iii) OTX2. Furthermore, the order can be (i) OTX2, (ii) NEUROD1 and (iii) GON4L or (i) OTX2, (ii) GON4L and (iii) NEUROD1. Also, the order can be (i) NEUROD1, (ii) GON4L and (iii) OTX2 or (i) NEUROD1, (ii) OTX2 and (iii) GON4L.

Also, one of the factors may be provided first before the two other factors are provided at about the same time, for example GON4L before OTX2 and NEUROD1, or OTX2 before GON4L and NEUROD1, or NEUROD1 before GON4L and OTX2.

The time frame between provision of a first, second, third and/or further TF or other factor that may be provided in the context of the method of the invention may be in the range of about 10, 15, 20, 25, 30, 40, 50 and/or 60 minutes. It may also be in the range of about 1, 1.5, 2, 3, 4, 5, 6, 7, 8, 9, 10, 11, 12, 13, 14, 15, 16, 17, 18, 19, 20, 21, 22, 23 and/or 24 hours. In embodiments the time frame between provision of TFs and/or other factors in the context of the method of the invention may be 1, 2, 3, 4, 5, 6, 7, 8, 9, 10, 11, 12, 13, 14, 15, 16, 17, 18, 19 and/or 20 days.

In embodiments of the invention, the method comprises the provision of micro-RNAs to the initial cell, preferably, miR-182 and/or miR-183.

In further embodiments, the one or more TFs and/or one or more micro-RNA, such as miR-182 and/or miR-183, are expressed from one or more exogenous nucleic acid molecules within the initial cell, wherein expression form the external nucleic acid results preferably in a level greater than present in the initial cell, for example a human iPSC.

In another embodiment of the invention, the initial cell is provided with one or more TFs and potentially other factors, such as miR-182 and/or miR-183 for at least 4 days, preferably about 7 to 10 days. In embodiments, provision with the one or more TFs and potentially other factors for only about 1 day is sufficient to induce a reprogramming of the initial cell to an induced photoreceptor cell, even if the photoreceptor phenotype may only occur after a further time frame.

Provision of GON4L and potentially the other factors, such as OTX2 and NEUROD1, for only a short initial time, such as one day, can be sufficient to induce a transdifferentiation program in the initial cell to develop into an induced photoreceptor cell, even if the initial external provision of the one or more TFs only occurred for a short period of time, such as 1 day. In embodiments of the invention, the initial cell is provided with one or more TFs and potentially other factors, such as miR-182 and/or miR-183 for at least about 0.25, 0.5, 0.75, 1.5, 2, 2.5, 3, 3.5, 4, 5, 6, 7, 8, 9, 10, 11, 12, 13, 14, 15, 16, 17, 18, 19, 20 days. Different factors provided during performing the method of the invention can be provided for different time periods and can be provided sequentially.

In embodiments of the invention, the one or more TFs and potentially micro-RNAs, such as miR-182 and/or miR-183, are expressed in the initial cell from one or more viral vectors, preferably lentiviral vectors.

In further embodiments, the one or more TFs and potentially micro-RNAs, such as miR-182 and/or miR-183, are provided by microinjection, transfection, electroporation of the factors and/or exogenous nucleic acid molecules for expression of the factors, for example transfection or electroporation of mRNA molecules.

In embodiments, the one or more TFs and potentially micro-RNAs, such as miR-182 and/or miR-183, are provided by a PiggyBac (PB) transposon system or other transposon systems. Such transposon systems are advantageous since they represent in safe method of factor delivery to an initial cell since the genetic elements can be removed from the cells after transient expression of for example the one or more TFs.

In preferred embodiments, the one or more TFs and potentially micro-RNAs, such as miR-182 and/or miR-183, are expressed transiently and/or expression is induced in the initial cell.

Embodiments with transient and/or induced provision or expression of the factors are particularly advantageous since after transient and/or induced expression or provision of the factors and induction of a differentiation program leading to differentiation of an induced photoreceptor cell or generation of an induced photoreceptor cell the external provision of the factors can be ended and the photoreceptor phenotype of the cells can be maintained by the expression endogenous factors and/or factors provided by the cellular environment. After withdrawal of the provided factors from the induced photoreceptor cells, these cells may behave more physiologically, since there is no forced external provision of factors. Therefore, the cells may resemble more to naturally occurring photoreceptor cells after withdrawal of the factors.

In embodiments of the invention, inducible expression is mediated by tetracycline-dependent transcriptional control. Expression of the one or more TFs and potentially micro-RNAs, such as miR-182 and/or miR-183, by means of tetracycline-controlled transcriptional activation is advantageous since tetracycline or one of its derivatives, e.g. doxycycline, can be easily provided to and also be withdrawn from the initial cell for controlling expression of TF from an exogenous nucleic acid molecule.

In further embodiments, the method of the invention comprises administering a cell cycle inhibitor to the initial cell, preferably AraC. Inhibitors, such as cell cycle inhibitors, are considered factors that can be provided to the cells during the method of the invention. Such inhibitors may be simply added to the cell culture medium during the method of the invention at a certain time point. For such inhibitors, the same time frames and criteria of for example sequential provision and time frames of provision apply as outlined above for transcription factors and potentially micro-RNAs. The use of cell cycle inhibitors during the method of the invention can be particularly advantageous when provided after one or more TF that initiates a reprogramming of the initial cell.

In embodiments, the cell cycle inhibitor is administered after providing the one or more TFs to the initial cell, preferably 5 days after providing the one or more TFs. In embodiments, the cell cycle inhibitor is administered to the initial cell 0.25, 0.5, 0.75, 1.5, 2, 2.5, 3, 3.5, 4, 5, 6, 7, 8, 9, 10, 11, 12, 13, 14, 15, 16, 17, 18, 19 or 20 days after providing the one or more TFs.

In embodiments of the method of the invention, the initial cells are cultivated on a basement membrane-like matrix, such as for example Matrigel or another gelatinous protein mixtures, such as specific collagen or laminin molecules that support development or maintenance of photoreceptor cells, such as poly-L-Lysine and poly-D-Lysine.

In some embodiments, the method comprises co-cultivation of the initial cells with retinal pigment epithelial cells (RPE-cells). Such embodiments of the invention are particularly advantageous since RPE-cells provide a cellular environment that promotes differentiation of the initial cells to induced photoreceptor cells.

### Embodiments Relating to the Detection of Induced Photoreceptors

In embodiments of the method of the invention, an induced photoreceptor cell produced from the initial cell is determined by a photoreceptor reporter system present in the initial cell, said reporter system preferably comprising one or more photoreceptor-specific promoter sequences, such as sequences from the arrestin- and/or rhodopsin-promoter, and one or more reporter genes and/or selection markers, such as a fluorescent protein gene.

The use of a photoreceptor reporter system in the context of the present invention is advantageous since it indicates the occurrence of a photoreceptor-phenotype in the initial cell and therefore can provide guidance as to whether and which of the induced cells can be used for downstream applications. Furthermore, the use of fluorescent reporter genes, such as GFP, RFP, dsRed and so on allows the detection of a photoreceptor phenotype by different methodologies, including microscopy and flow cytometry. The use of several different promoter sequence with different specificities further allows a specification of the phenotype, for example simultaneous use of cone- and rod-specific promoter sequences with different reporter genes, such as genes encoding for fluorescent proteins of different color, allows detection and subsequent isolation of rod-like and cone-like cells in a mixed culture of the method of the invention. For example, the rhodopsin is a rod-specific protein and therefore activity of the rhodopsin-promoter indicates development of a rod-like phenotype. In contrast, certain arrestin proteins, such as arrestin-3, are cone specific and their promoter can be used in the context of the invention to monitor cone-development. Furthermore, selection markers such as genes that render cells resistant to certain toxic chemicals such as antibiotics can be expressed under the control of a photoreceptor-specific promoter to select induced photoreceptors from a mixed cell culture.

In embodiments of the invention the induced photoreceptor cells are isolated from the cell culture that may comprise uninduced initial cells or other cells than induced photoreceptors. The isolation of induced photoreceptors can occur through use of marker genes or proteins such as fluorescent proteins, for example through fluorescence activated cell sorting. Also, isolation of induced photoreceptor cells may occur through magnetic cell separation FACS-sorting, for example on the basis of surface marker expression. Furthermore, isolation can occur through expression selection markers making the cells resistant to chemical compounds, so that all non-induced cells disappear from the culture. A skilled person is able to use further techniques known in the art to separate induced photoreceptor cells from a cell culture system comprising further cell types.

In further embodiments of the invention, it may be useful to generate monoclonal or clonal cell lines from the induced photoreceptor cells.

In embodiments of the invention, generation of an induced photoreceptor cell is determined by expression of genes or proteins that are specifically expressed in photoreceptor cells, but not in the initial cells of the method of the invention. In some embodiments, the marker molecules described below are expressed in greater amounts in induced photoreceptors compared to the initial cell, such as iPSC. In embodiments of the invention, generation of an induced photoreceptor cell is determined by expression of endogenous recoverin, NCAM, OTX, CRX, RCVRN, RHO, OPN1SW and/or OPN1LW. Detection of expression can occur on a protein or mRNA level, for example by qPCR, antibody-mediated detection methods and other well-established techniques known to the person skilled in the art.

In embodiments, generation of an induced photoreceptor cell is determined through formation of neurite outgrowth in an in vitro assay. Neurite outgrowth are an indicator of a neuronal phenotype, which indicates the induction of neuroepithelial photoreceptors.

In embodiments, the initial cell is an iPSC and generating an induced photoreceptor cell is determined through loss of Tra1-60 expression. Tra1-60 is a iPSC marker that disappears from the initial cells upon induction of a photoreceptor phenotype.

### Embodiments Relating to the Induced Photoreceptor Cells

The present invention further relates to an induced photoreceptor cell produced by the method of the present invention.

Furthermore, the invention relates to an induced photoreceptor cell obtainable by the method of the present invention. Accordingly, the invention relates to all kinds of induced photoreceptor cells that display an identical phenotype as the cells generated by the method of the invention, such as a specific gene expression signature, combination of surface markers, cellular shape and/or cellular function, which is different form naturally occurring photoreceptor cells and induced photoreceptor cells generated through methods of the state of the art.

The cells that are generated by the method of the present invention may not necessarily be best described by structural features, because for example the method of the invention can be performed by using different means of providing the TF of the present invention. For example, the TF may be provided by means of exogenous nucleic acids. However, these nucleic acids may disappear (be transient) from the cells later on. Accordingly, the cells cannot necessarily be defined by the presence of an exogenous nucleic acid. Similarly, in the context of the method of the invention, after provision of the TF, the cells can initiate expression of the endogenous TF, so that after a certain period it may not be possible to detect exogenous TF anymore, while the cells maintain the induced photoreceptor phenotype by expression of endogenous factors. However, a skilled person is able to analyze the cells of the present invention generated by the method of the invention, for example by analysis of their global expression profile, in order to distinguish them from physiologically occurring photoreceptor cells.

In some embodiment, the methods described herein induce photoreceptor cells that display the features of the induced photoreceptors described in the examples. The present invention further relates to the induced photoreceptor cells described in the examples disclosed herein. The cells of the present invention express at least one of the photoreceptor markers measured in the examples, including NCAM, OTX2, CRX, RCVRN, RHO, mCAR, OPNISW and OPNILW. In some embodiments, the induced photoreceptors of the invention do not express SOX2 or OCT4. In this context, in some embodiments, the expression of the respective factors may be judged by comparing the expression in the cells of the invention to the expression level of the respective factors in iPSC. For example, in some embodiments, lack of OCT4 or SOX2 can relate to an expression level that is about 10% or less than the expression in iPSC. On the contrary, in some embodiments, expression of the photoreceptor markers in the cells of the invention can be about 10 times higher than in iPSC.

The invention further relates to induced photoreceptor cells comprising at least one exogenous nucleic acid molecule comprising a sequence encoding GON4L, preferably under the control of a suitable regulatory nucleic acid sequence, such as a constitutive or inducible promoter or promoter/enhancer combination. The cells of the present invention may be generated using a kit of the invention as disclosed herein. In general, the features disclosed in the context of a method or kit of the invention also relate to and are herewith disclosed in the context of the cells of the present invention and vice versa.

The cells of the invention can be used for research and development purposes, for example for identifying, testing and screening of potential drugs affecting or acting on photoreceptor cells.

In addition, the invention relates to the cells of the invention for use as a medicament in the treatment of retinopathy, such as retinal degeneration. The cells can be transplanted to the retina of affected patients. The transplanted cells of the invention may be at different differentiation stages. For example, the induced photoreceptor cells may be at a photoreceptor precursor stage at the time point of transplantation and develop into mature photoreceptors after transplantation into the retina. Alternatively, or in addition, more mature or mature induced photoreceptor cells may be transplanted; however, this may depend on the individual patient needs and conditions.

It is a great advantage of the method of the invention that it enables provision of patient specific induced photoreceptor cells that may be used as a medicament in the treatment of the patient. Furthermore, through use of HLA-matched iPSC from iPSC banks enables provision of suitable induced photoreceptor cells generated from matching iPSC to a patient. This is particularly advantageous if the condition leading to the necessity of induced photoreceptor cell transplantation or treatment is genetic, because it is possible to provide matching photoreceptor cells without relying of the patients own cell donation, which may require correction of the genetic cause leading to the disease necessitating photoreceptor transplantation.

### Further Embodiments of the Invention

The present invention also relates to a kit for producing induced photoreceptor cells from an initial cell according to the method disclosed herein. A kit of the invention comprises:
a. a vector system for providing GON4L, and optionally further TFs, preferably OTX2 and/or NEUROD1, and optionally miR-182 and/or miR-183 to the initial cell,
b. reagents for detecting induced photoreceptor cells generated from an initial cell, such as
   i. a photoreceptor-specific reporter system,
   ii. antibodies for detection of photoreceptor marker proteins, e.g. OPNISW, OPN1 LW, recoverin and/or NCAM, and/or
   iii. primers for detection of OTX, CRX, RCVRN, RHO, OPN1SW, OPN1MW and/or OPN1LW mRNA by PCR, and
c. optionally a cell cycle inhibitor, preferably AraC.

Furthermore, the invention relates to an expression vector system comprising one or more nucleic acid sequences operably coupled to one or more promoters, wherein said sequences encode one or more transcription factors (TFs) comprising at least GON4L, OTX2 and NEUROD1, and optionally miR-182 and/or miR-183. Preferred embodiments of vectors are provided below.

The present invention also relates to a transcription factor combination comprising at least GON4L, OTX2 and NEUROD1. The combination may relate to a combination of TFs in protein form, a combination of TF encoding nucleic acids, the combination of TF encoding nucleic acids in a vector or other expressible format, or the combination of these TFs above levels of the initial cell, such as the iPSC, in a modified cell.

### DETAILED DESCRIPTION OF THE INVENTION

All cited documents of the patent and non-patent literature are hereby incorporated by reference in their entirety.

The present invention is directed to a method for producing induced photoreceptor cells from an initial cell, the method comprising providing one or more transcription factors (TFs) comprising at least GON4L to the initial cell.

In the context of the invention the term induced photoreceptor cell relates to a cell with a phenotype resembling to a naturally occurring photoreceptor cell or a progenitor of such a photoreceptor cell, wherein the induced photoreceptor cell has developed or differentiated from an initial cell that is not a photoreceptor cell. An induced photoreceptor cell displays characteristics of photoreceptor cells and progenitors of photoreceptor cells such as expression of one or more genes and proteins that are specific to photoreceptor and their progenitors, at least in combination with each other, and/or display a photoreceptor-like morphology including neurite outgrowths. Such markers include without limitation recoverin (RCVRN), rhodopsin, cone-arrestin (arrestin-4), arrestin-1, NCAM, CRX, NEUROD1, NR2E1, NR2E3, NRL1, OTX2, ONECUT1, PAX6, RAX, RORB, RXRG, SIX3, SIX6, SOX2, THRB, VSX2, OTX, RHO, OPNISW, OPN1 MW and/or OPN1LW. Expression of such markers may exist to some extent in other cell types; however, such markers may be well known for being involved in photoreceptor differentiation. Furthermore, the development or induction of a photoreceptor cell from an initial cell may be monitored or detected by the absence of a marker of the initial cell. The absolute absence of such a repressed marker of the initial cell is not required for “repression” according to the present invention. It is possible that low levels remain present in the cell. Repression of markers of the initial cell may be characterised as reduced levels of expression compared to the initial cell. Reduced levels compared to an appropriate control may be used for determining “repression”. Similarly, “activation” of gene expression of photoreceptor-specific genes can be determined by comparison to an appropriate control, such as the respective initial cell. Induced photoreceptors are characterized by their transcriptional profiles, which can be derived from a bulk population or from single cell RNA sequencing analysis. Such profiles can be used to differentiate induced photoreceptor cell of the present invention from naturally occurring photoreceptor cells.

Photoreceptor cells are a specialized type of neuroepithelial cell found in the retina that is capable of visual phototransduction. Photoreceptors convert light (visible electromagnetic radiation) into signals that can stimulate biological processes. Photoreceptor proteins in these cells absorb photons, triggering a change in the cell's membrane potential. Mammalian photoreceptor cells include rods, cones, and photosensitive retinal ganglion cells. The two classic photoreceptor cells are rods and cones. The rods are narrower than the cones and distributed differently across the retina, but the chemical process in each that supports phototransduction is similar. The photosensitive ganglion cells do not contribute to sight directly but are thought to support circadian rhythms and pupillary reflex.

Rods are extremely sensitive and can be triggered by a single photon. At very low light levels, visual experience is based solely on the rod signal, so that colors cannot be seen at low light levels. Cones require significantly brighter light (i.e., a larger number of photons) in order to produce a signal. In humans, there are three different types of cone cell, distinguished by their pattern of response to different wavelengths of light. Color experience is calculated from these three distinct signals. The three types of cone cell respond (roughly) to light of short, medium, and long wavelengths. The human retina contains about 120 million rod cells, and 6 million cone cells. The number and ratio of rods to cones varies among species, dependent on whether an animal is primarily diurnal or nocturnal. In the human visual system, in addition to the photosensitive rods & cones, there are about 2.4 million to 3 million ganglion cells, with 1 to 2% of them being photosensitive. The axons of ganglion cells form the two optic nerves.

The method of the invention relates to providing one or more transcription factors. Providing a transcription factor or other factor, such as a micro-RNA, in the context of the present invention relates to provision or making available or contacting a TF with the initial cell or introducing the TF within the cell, or having the TF produced from within or in close proximity to the initial cell. The TF may be provided at the protein level or in the form of a nucleic acid encoding a TF. Accordingly, in case of delivery of an exogenous nucleic acid molecule encoding the TF, the TF is provided upon expression of the protein from the exogenous nucleic acid molecule. A TF can be provided through expression from any given nucleic acid molecule. This includes activation of expression of the respective TF from an endogenous or an exogenous nucleic acid molecule. Furthermore, the TF can be delivered to the cell directly, for example by protein transfection. Preferably, the expression of a TF occurs in amounts greater than the initial cell, e.g. iPSCs.

TF provision can occur by expression from a nucleic acid molecule, such as an exogenous nucleic acid molecule. As used herein “nucleic acid” shall mean any nucleic acid molecule, including, without limitation, DNA, RNA and hybrids or modified variants thereof. An “exogenous nucleic acid” or “exogenous genetic element” relates to any nucleic acid introduced into the cell, which is not a component of the cells “original” or “natural” genome. Exogenous nucleic acids may be integrated or non-integrated in the genetic material of the target mesenchymal stem cell or relate to stably transduced nucleic acids. Delivery of an exogenous nucleic acid may lead to genetic modification of the initial cell through permanent integration of the exogenous nucleic acid molecule in the initial cell. However, delivery of the exogenous nucleic acid may also be transient, meaning that the delivered genetic material for provision of the one or more TF disappears form the cell after a certain time.

Nucleic acid molecule delivery and potentially genetic modification of an initial cell, such as a mammalian or human cell, preferably a human iPSC, can be performed and determined by a skilled person using commonly available techniques. For example, for detecting genetic modification sequencing of the genome or parts thereof of an initial cell is possible, thereby identifying if exogenous nucleic acids are present. Alternatively, other molecular biological techniques may be applied, such as the polymerase chain reaction (PCR), to identify/amplify exogenous genetic material. Exogenous nucleic acids may be detected by vector sequences, or parts of vector sequences remaining at the site of genetic modification. In cases where vector sequences (for example vector sequences flanking a therapeutic transgene) can be removed from the genome, the addition of a therapeutic transgene may still be detected by sequencing efforts by detecting genomic sequences incorporating a therapeutic gene at a “non-natural” position in the genome.

Any given gene delivery method for delivery of nucleic acid molecules is encompassed by the invention and preferably relates to viral or non-viral vectors, as well as biological or chemical methods of transfection. The methods can yield either stable or transient gene expression in the system used. Furthermore, any method known to the person skilled in the art for delivery of proteins to a mammalian cell is encompassed by the present invention when referring to provision of one or more transcription factors and/or micro-RNAs or other factors. All known methods for delivery of nucleic acid molecules and proteins as well as other biological and chemical molecules that can act as factors in the context of the method of the invention are encompassed. This includes in particular microinjection, transfection, transduction, vesicle fusion and electroporation.

Genetically modified viruses have been widely applied for the delivery of genes into mammalian cells and in particular stem cells. A viral vector may be employed in the context of the present invention.

Preferred viral vectors for genetic modification of the initial cells described herein relate to retroviral vectors, in particular to gamma retroviral vectors. The gamma retrovirus (sometimes referred to as mammalian type C retroviruses) is a sister genus to the lentivirus clade, and is a member of the Orthoretrovirinae subfamily of the retrovirus family. The Murine leukemia virus (MLV or MuLV), the Feline leukemia virus (FeLV), the Xenotropic murine leukemia virus-related virus (XMRV) and the Gibbon ape leukemia virus (GALV) are members of the gamma retrovirus genus. A skilled person is aware of the techniques required for utilization of gamma retroviruses in genetic modification of MSCs. For example, the vectors described Maetzig et al (Gammaretroviral vectors: biology, technology and application, 2001, Viruses June; 3(6):677-713) or similar vectors may be employed. For example, the Murine Leukemia Virus (MLV), a simple gammaretrovirus, can be converted into an efficient vehicle of genetic therapeutics in the context of creating gamma retrovirus-modified MSCs and expression of a therapeutic transgene from said MSCs after delivery to a subject.

Lentiviruses are members of Retroviridae family of viruses (M. Scherr et al., Gene transfer into hematopoietic stem cells using lentiviral vectors. Cuff Gene Ther. 2002 February; 2(1):45-55). Lentivirus vectors are generated by deletion of the entire viral sequence with the exception of the LTRs and cis acting packaging signals. The resultant vectors have a cloning capacity of about 8 kb. One distinguishing feature of these vectors from retroviral vectors is their ability to transduce dividing and non-dividing cells as well as terminally differentiated cells.

The invention encompasses further the administration of expression vectors to a subject in need thereof. A “vector” is any means for the transfer of a nucleic acid into a host cell. A preferred vector relates to a replicon to which another DNA segment may be attached so as to bring about the replication of the attached segment. The term “vector” as used herein specifically refers to means for introducing the nucleic acid into a cell in vitro, ex vivo or in vivo. Viral vectors include, without limitation, retrovirus, adeno-associated virus, pox, baculovirus, vaccinia, herpes simplex, Epstein-Barr and adenovirus vectors.

Adenoviruses may be applied, or RNA viruses such as Lentiviruses, or other retroviruses. Adenoviruses have been used to generate a series of vectors for gene transfer cellular engineering. The initial generation of adenovirus vectors were produced by deleting the EI gene (required for viral replication) generating a vector with a 4 kb cloning capacity. An additional deletion of E3 (responsible for host immune response) allowed an 8 kb cloning capacity. Further generations have been produced encompassing E2 and/or E4 deletions.

Non-integrating viral systems, such as adeno-associated viral vectors (AAV), represent a preferred embodiment for the gene therapy approaches described herein due to a number of advantageous benefits (see Asokan et al., Molecular Therapy vol. 20 no. 4, 699-708). For example, AAV are of particular interest in gene therapy due to their very limited capacity to induce immune responses in humans, a factor which positively influences vector transduction efficiency while reducing the risk of any immune-associated pathology. The AAV genome is typically built of single-stranded deoxyribonucleic acid (ssDNA), either positive- or negative-sensed, which is about 4.7 kilobases long. The AAV genome comprises inverted terminal repeats (ITRs) at both ends of the DNA strand, and two open reading frames (ORFs): rep and cap. Development of AAVs as gene therapy vectors has eliminated the integrative capacity of the vector by removal of the rep and cap from the DNA of the vector. Any given desired gene, together with a promoter to drive transcription of the gene (for example the inventive TGIF2 as described herein), is inserted between the inverted terminal repeats (ITR) that aid concatamer formation in the nucleus after the single-stranded vector DNA is converted by host cell DNA polymerase complexes into double-stranded DNA. AAV-based gene therapy vectors typically form episomal concatamers in the host cell nucleus. In non-dividing cells, these concatemers remain intact for the life of the host cell. In dividing cells, AAV DNA is lost through cell division, since the episomal DNA is not replicated along with the host cell DNA.

As regards viruses, these are preferably previously purified (e.g., by centrifugation on a cesium chloride gradient, column chromatography, plaque purification, and the like). They may be packaged at the rate of 10⁴ to 10¹⁵ particles per ml, preferably 10⁵ to 10¹².

Non-viral methods may also be employed, such as alternative strategies that include conventional plasmid transfer and the application of targeted gene integration through the use of integrase or transposase technologies. These represent approaches for vector transformation that have the advantage of being both efficient, and often site-specific in their integration. Physical methods to introduce vectors into cells are known to a skilled person. One example relates to electroporation, which relies on the use of brief, high voltage electric pulses which create transient pores in the membrane by overcoming its capacitance. One advantage of this method is that it can be utilized for both stable and transient gene expression in most cell types. Alternative methods relate to the use of liposomes or protein transduction domains. Appropriate methods are known to a skilled person and are not intended as limiting embodiments of the present invention. Furthermore, delivery of RNA molecules such as mRNA transfection is included in the context of the method of the invention for provision of a TF from an exogenous nucleic acid.

Furthermore, delivery of exogenous nucleic acid molecules for provision of a factor may be achieved by means of a transposable element. For example, the Sleeping Beauty, Tol2 and/or

PiggyBac transposon system or similar systems may be used. The PiggyBac (PB) transposon is a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and efficiently integrates them into TTAA chromosomal sites. The powerful activity of the PiggyBac transposon system enables genes of interest between the two ITRs in the PB vector to be easily mobilized into target genomes. The TTAA-specific transposon PiggyBac is a highly useful transposon for genetic engineering of a wide variety of cells, including mammalian and human cells, in particular stem cells and iPSC.

Provision of the TFs and other factors used in the method of the invention may be transient or permanent. For example, if provision is achieved by expression from a nucleic acid molecule, TF expression may be permanently active under the control of a constitutive promoter or a promoter that is active in the initial cell as well as in induced photoreceptor cells. Alternatively, expression and therefore provision of the TF may be transient, either because the nucleic acid molecule that encodes the TF is removed or disappears from the cell or because expression is controllable and can be turned on and off, for example by using controlled transcriptional activation. In the context of the present invention, transient expression refers to only temporal expression of a factor from a nucleic acid molecule in contrast to permanent expression. Transient expression can be based on expression from a delivered mRNA molecule, which gets degraded over time in the cell and therefore expression only occurs as long as the delivered mRNA has not been degraded.

Transient expression can in other examples occur through induction of gene expression from an exogenous DNA molecule comprising controllable genetic elements driving expression of the encoded gene, and therefore comprises inducible gene expression. In such systems gene expression can be externally controlled, for example through administration of a compound, such as a chemical compound, for example an antibiotic molecule or drug that leads to activation of gene expression. Such systems are well described in the art and are known to the skilled person.

A gene expression system that may be used in the context of the invention is a system specifically designed for the production of a gene product of choice. This is normally a protein although may also be RNA, such as micro-RNA. An expression system consists of a gene, normally encoded by DNA, and the molecular machinery required to transcribe the DNA into mRNA and translate the mRNA into protein using the reagents provided. An expression system is therefore often artificial in some manner; however, certain parts of the machinery required for gene expression may be provided by the target cell.

For example, inducible and/or controlled gene expression can be achieved by the use of tetracycline-controlled transcriptional activation. Tetracycline-Controlled Transcriptional Activation is a method of inducible gene expression where transcription is reversibly turned on or off in the presence of the antibiotic tetracycline or one of its derivatives (e.g. doxycycline). Tetracycline-controlled gene expression is based upon the mechanism of resistance to tetracycline antibiotic treatment found in Gram-negative bacteria, where the Ptet promoter expresses TetR, the repressor, and TetA, the protein that pumps tetracycline antibiotic out of the bacterial cell. The difference between a Tet-On and Tet-Off system is not whether the transactivator turns a gene on or off, but rather, both proteins activate expression. The difference relates to their respective response to tetracycline or doxycycline (Dox, a more stable tetracycline analogue); Tet-Off activates expression in the absence of Dox, whereas Tet-On activates in the presence of Dox.

In the context of the invention the term transcription factor (TF) relates to a protein that controls the rate of transcription of genetic information from DNA to messenger RNA, by binding to a specific DNA sequence. The function of TFs is to regulate (turn on and off) genes in order to make sure that they are expressed in the right cell at the right time and in the right amount throughout the life of the cell and the organism. Groups of TFs function in a coordinated fashion to direct cell differentiation, cell division, cell growth, and cell death throughout life; cell migration and organization (body plan) during embryonic development; and intermittently in response to signals from outside the cell, such as a hormone. TFs work alone or with other proteins in a complex, by promoting (as an activator), or blocking (as a repressor) the recruitment of RNA polymerase (the enzyme that performs the transcription of genetic information from DNA to RNA) to specific genes. A defining feature of TFs is that they contain at least one DNA-binding domain (DBD), which attaches to a specific sequence of DNA adjacent to the genes that they regulate.

Transcription factors can be used for reprogramming or directed differentiation of mammalian cells to a different cell type. Induction of a different cell type in an initial cell/staring cell can be achieved through provision of one or more TF. In the context of the present invention, the term “initial cell” relates to a cell that is used as a starting point for inducing a photoreceptor phenotype in this cell, wherein at least the TF GON4L is provided in the cell. In the context of the invention, any kind of cell, preferably a mammalian cell can be used as an initial cell. Preferably the initial cell is a human cell. A cell is the basic structural, functional, and biological unit of all known living organisms. A cell is the smallest unit of life. Cells are often called the “building blocks of life”.

Preferable initial cells of the present invention are pluripotent or multipotent mammalian cells, including stem cells. Preferably the initial cell is a mammalian, preferably a human induced pluripotent stem cell (iPSC). iPSCs are a type of pluripotent stem cell that can be generated directly from adult cells. iPSC can propagate indefinitely in cell culture, as well as give rise to every other cell type in the body or the respective mammalian organism, such as the human organism, including neurons, heart cells, pancreatic cells, and liver cells, they represent a single source of cells that could be used to replace those lost to damage or disease. The most well-known type of pluripotent stem cell is the embryonic stem cell. However, since the generation of embryonic stem cells involves manipulation of the pre-implantation stage embryo, there has been much ethical controversy surrounding their use. Further, because embryonic stem cells can only be derived from embryos, it has so far not been feasible to create patient-matched embryonic stem cell lines. Since iPSCs can be derived directly from adult tissues, they not only bypass the need for embryos, but can be made in a patient-matched manner, which means that each individual could have their own pluripotent stem cell line. These unlimited supplies of autologous cells could be used to generate transplants without the risk of immune rejection. Furthermore, iPSC and iPSC derived cells can be used in personalized drug discovery efforts and understanding the patient-specific basis of disease. This also applies to the induced photoreceptor cells of the present invention that can be derived from human patient specific iPSC. iPSCs are typically derived by introducing products of specific sets of pluripotency-associated genes, or “reprogramming factors”, into a given cell type. The original set of reprogramming factors are the transcription factors Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers. Such replacement of factors required for cellular reprogramming also applies to other cellular reprogramming efforts.

Further initial cells to be used in the context of the present invention are fibroblasts, retinal progenitor cell (RPCs), retinal pigment epithelium (RPE) cells, Mueller Glia cells and other cell types found in the eye or retina that are no photoreceptors in the sense of the present invention.

The method of the invention includes the provision of the TF GON4L to the initial cell. GON4L is a protein that in humans is encoded by the GON4L gene. It is a nuclear protein containing two serine phosphosites and a lysine-glutamine cross-link and is thought to be a transcription factor. Homologs of GON4L have conserved roles in cell cycle regulation and/or embryonic patterning in plants, worms, flies, mice, and fish. However, the contribution of GON4L or any other chromatin factor to morphogenesis is particularly poorly understood.

Furthermore, the present invention preferably relates to the provision of one or more TFs selected from CRX, NEUROD1, NR2E1, NR2E3, NRL1, OTX2, ONECUT1, PAX6, RAX, RORB, RXRG, SIX3, SIX6, SOX2, THRB and VSX2. These TFs have been described to be highly relevant during differentiation and development of photoreceptor cells.

OTX2 is a protein that in humans is encoded by the OTX2 gene. This gene encodes a member of the bicoid sub-family of homeodomain-containing transcription factors. The encoded protein acts as a transcription factor and may play a role in brain and sensory organ development. A similar protein in mice is required for proper forebrain development. Two transcript variants encoding distinct isoforms have been identified for this gene. Other alternative splice variants may exist, but their full-length sequences have not been determined.

NEUROD1/NeuroD1 (Neurogenic differentiation 1), also called 02, is a transcription factor of the NeuroD-type. It is encoded by the human gene NEUROD1. It is a member of the NeuroD family of basic helix-loop-helix (bHLH) transcription factors. The protein forms heterodimers with other bHLH proteins and activates transcription of genes that contain a specific DNA sequence known as the E-box. It regulates expression of the insulin gene, and mutations in this gene result in type II diabetes mellitus. NeuroD1 is found to convert reactive glial cells into functional neurons in the mouse brain in vivo.

In the context of the invention, the one or more TF may be provided at the protein level or in the form of a nucleic acid encoding a TF.

Preferred amino acid sequences of GON4L, NEUROD1 and OTX2 are listed under Table 1.

TABLE 1
Amino acid sequences of preferred TF of the invention.

| | |
|---|---|
| SEQ ID NO 1: | MYPELLPVCSLKAKNPQDKIVFTKAEDNLLALGLKHFEGTEFPNP |
| Amino acid (AA) sequence | LISKYLLTCKTAHQLTVRIKNLNMNRAPDNIIKFYKKTKQLPVLGKC |
| of human GON4L protein | CEEIQPHQWKPPIEREEHRLPFWLKASLPSIQEELRHMADGARE |
| GenBank: AAI17558.1 | VGNMTGTTEINSDRSLEKDNLELGSESRYPLLLPKGVVLKLKPVA |
| | TRFPRKAWRQKRSSVLKPLLIQPSPSLQPSFNPGKTPARSTHSE |
| | APPSKMVLRIPHPIQPATVLQTVPGVPPLGVSGGESFESPAALPA |
| | VPPEARTSFPLSESQTLLSSAPVPKVMLPSLAPSKFRKPYVRRRP |
| | SKRRGVKASPCMKPAPVIHHPASVIFTVPATTVKIVSLGGGCNMI |
| | QPVNAAVAQSPQTIPITTLLVNPTSFPCPLNQSLVASSVSPLIVSG |
| | NSVNLPIPSTPEDKAHVNVDIACAVADGENAFQGLEPKLEPQELS |
| | PLSATVFPKVEHSPGPPLADAECQEGLSENSACRWTVVKTEEG |
| | RQALEPLPQGIQESLNNPTPGDLEEIVKMEPEEAREEISGSPERDI |
| | CDDIKVEHAVELDTGAPSEELSSAGEVTKQTVLQKEEERSQPTK |
| | TPSSSQEPPDEGTSGTDVNKGSSKNALSSVDPEVRLSSPPGKPE |
| | DSSSVDGQSVGTPVGPETGGEKNGPEEEEEEDFDDLTQDEEDE |
| | MSSASEESVLSVPELQETMEKLTWLASERRMSQEGESEEENSQ |
| | EENSEPEEEEEEEAEGMESLQKEDEMTDEAVGDSAEKPPTFAS |
| | PETAPEVETSRTPPGESIKAAGKGRNNHRARNKRGSRARASKDT |
| | SKLLLLYDEDILERDPLREQKDLAFAQAYLTRVREALQHIPGKYED |
| | FLQVIYEFESSTQRRTAVDLYKSLQILLQDWPQLLKDFAAFLLPEQ |
| | ALACGLFEEQQAFEKSRKFLRQLEICFAENPSHHQKIIKVLQGCA |
| | DCLPQEITELKTQMWQLLKGHDHLQDEFSIFFDHLRPAASRMGD |
| | FEEINWTEEKEYEFDGFEEVALPDVEEEEEPPKIPTASKNKRKKEI |
| | GVQNHDKETEWPDGAKDCACSCHEGGPDSKLKKSKRRSCSHC |
| | SSKVRKVSRVPRVSELLGDCLLPRIVPY |
| SEQ ID NO 2: | MLPCKKRRTTVTESLQHKGNQEENNVDLESAVKPESDQVKDLS |
| Amino acid (AA) sequence | SVSLSWDPSHGRVAGFEVQSLQDAGNQLGMEDTSLSSGMLTQ |
| of human GON4L isoform A | NTNVPILEGVDVAISQGITLPSLESFHPLNIHIGKGKLHATGSKRGK |
| GenBank: AAR01260.1 | KMTLRPGPVTQEDRCDHLTLKEPFSGEPSEEVKEEGGKPQMNS |
| | EGEIPSLPSGSQSAKPVSQPRKSTQPDVCASPQEKPLRTLFHQP |
| | EEEIEDGGLFIPMEEQDNEESEKRRKKKKGTKRKRDGRGQEGTL |
| | AYDLKLDDMLDRTLEDGAKQHNLTAVNVRNILHEVITNEHVVAM |
| | MKAAISETEDMPMFEPKMTRSKLKEVVEKGVVIPTWNISPIKKAN |
| | EIKPPQFVDIHLEEDDSSDEEYQPDDEEEDETAEESLLESDVEST |
| | ASSPRGAKKSRLRQSSEMTETDEESGILSEAEKVTTPAIRHISAE |
| | VVPMGPPPPPKPKQTRDSTFMEKLHAVDEELASSPVCMDSFQP |
| | MDDSLIAFRTRSKMPLKDVPLGQLEAELQAPDITPDMYDPNTAD |
| | DEDWKMWLGGLMNDDVGNEDEADDDDDPEYNFLEDLDEPDTE |
| | DFRTDRAVRITKKEVNELMEELFETFQDEMGFSNMEDDGPEEEE |
| | CVAEPRPNFNTPQALRFEEPLANLLNEQHRTVKELFEQLKMKKS |
| | SAKQLQEVEKVKPQSEKVHQTLILDPAQRKRLQQQMQQHVQLLT |
| | QIHLLATCNPNLNPEATTTRIFLKELGTFAQSSIALHHQYNPKFQT |
| | LFQPCNLMGAMQLIEDFSTHVSIDCSPHKTVKKTANEFPCLPKQV |
| | AWILATSKVFMYPELLPVCSLKAKNPQDKIVFTKAEDNLLALGLKH |
| | FEGTEFPNPLISKYLLTCKTAHQLTVRIKNLNMNRAPDNIIKFYKKT |
| | KQLPVLGKCCEEIQPHQWKPPIEREEHRLPFWLKASLPSIQEELR |
| | HMADGAREVGNMTGTTEINSDRSLEKDNLELGSESRYPLLLPKG |
| | VVLKLKPVATRFPRKAWRQKRSSVLKPLLIQPSPSLQPSFNPGKT |
| | PARSTHSEAPPSKMVLRIPHPIQPATVLQTVPGVPPLGVSGGESF |
| | ESPAALPAVPPEARTSFPLSESQTLLSSAPVPKVMLPSLAPSKFR |
| | KPYVRRRPSKRRGVKASPCMKPAPVIHHPASVIFTVPATTVKIVS |
| | LGGGCNMIQPVNAAVAQSPQTIPITTLLVNPTSFPCPLNQSLVAS |
| | SVSPLIVSGNSVNLPIPSTPEDKAHVNVDIACAVADGENAFQGLE |
| | PKLEPQELSPLSATVFPKVEHSPGPPLADAECQEGLSENSACRW |
| | TVVKTEEGRQALEPLPQGIQESLNNPTPGDLEEIVKMEPEEAREE |
| | ISGSPERDICDDIKVEHAVELDTGAPSEELSSAGEVTKQTVLQKE |
| | EERSQPTKTPSSSQEPPDEGTSGTDVNKGSSKNALSSMDPEVR |
| | LSSPPGKPEDSSSVDGQSVGTPVGPETGGEKNGPEEEEEEDFD |
| | DLTQDEEDEMSSASEESVLSVPELQETMEKLTWLASERRMSQE |
| | GESEEENSQEENSEPEEEEEEEAEGMESLQKEDEMTDEAVGDS |
| | AEKPPTFASPETAPEVETSRTPPGESIKAAGKGRNNHRARNKRG |
| | SRARASKDTSKLLLLYDEDILERDPLREQKDLAFAQAYLTRVREA |
| | LQHIPGKYEDFLQVIYEFESSTQRRTAVDLYKSLQILLQDWPQLLK |
| | DFAAFLLPEQALACGLFEEQQAFEKSRKFLRQLEICFAENPSHHQ |
| | KIIKVLQGCADCLPQEITELKTQMWQLLKGHDHLQDEFSIFFDHLR |
| | PAASRMGDFEEINWTEEKEYEFDGFEEVALPDVEEEEEPPKIPTA |
| | SKNKRKKEIGVQNHDKETEWPDGAKDCACSCHEGGPDSKLKKS |
| | KRRSCSHCSSKVCDSKSYKSKEPHELVGSSPHREASPMPGAKE |
| | AGQGKDMMEEEAPEERESTEATQSRTVRTTRKGEMPVSAGLAV |
| | GSTLPSPREVTVTERLLLDGPPPHSPETPQFPPTTGAVLYTVKRN |
| | QVGPEVRSCPKASPRLQKEREGQKAVSESEALMLVWDASETEK |
| | LPGTVEPPASFLSPVSSKTRDAGRRHVSGKPDTQERWLPSSRA |
| | RVKTRDRTCPVHESPSGIDTSETSPKAPRGGLAKDSGTQAKGPE |
| | GEQQPKAAEATVCANNSKVSSTGEKVVLWTREADRVILTMCQE |
| | QGAQPQTFNIISQQLGNKTPAEVSHRFRELMQLFHTACEASSED |
| | EDDATSTSNADQLSDHGDLLSEEELDE |
| SEQ ID NO 3: | MLPCKKRRTTVTESLQHKGNQEENNVDLESAVKPESDQVKDLS |
| Amino acid (AA) sequence | SVSLSWDPSHGRVAGFEVQSLQDAGNQLGMEDTSLSSGMLTQ |
| of human GON4L isoform B | NTNVPILEGVDVAISQGITLPSLESFHPLNIHIGKGKLHATGSKRGK |
| GenBank: AAR01262.1 | KMTLRPGPVTQEDRCDHLTLKEPFSGEPSEEVKEEGGKPQMNS |
| | EGEIPSLPSGSQSAKPVSQPRKSTQPDVCASPQEKPLRTLFHQP |
| | EEEIEDGGLFIPMEEQDNEESEKRRKKKKGTKRKRDGRGQEGTL |
| | AYDLKLDDMLDRTLEDGAKQHNLTAVNVRNILHEVITNEHVVAM |
| | MKAAISETEDMPMFEPKMTRSKLKEVVEKGVVIPTWNISPIKKAN |
| | EIKPPQFVDIHLEEDDSSDEEYQPDDEEEDETAEESLLESDVEST |
| | ASSPRGAKKSRLRQSSEMTETDEESGILSEAEKVTAPAIRHISAE |
| | VVPMGPPPPPKPKQTRDSTFMEKLHAVDEELASSPVCMDSFQP |
| | MDDSLIAFRTRSKMPLKDVPLGQLEAELQAPDITPDMYDPNTAD |
| | DEDWKMWLGGLMNDDVGNEDEADDDDDPEYNFLEDLDEPDTE |
| | DFRTDRAVRITKKEVNELMEELFETFQDEMGFSNMEDDGPEEEE |
| | CVAEPRPNFNTPQALRFEEPLANLLNEQHRTVKELFEQLKMKKS |
| | SAKQLQEVEKVKPQSEKVHQTLILDPAQRKRLQQQMQQHVQLLT |
| | QIHLLATCNPNLNPEATTTRIFLKELGTFAQSSIALHHQYNPKFQT |
| | LFQPCNLMGAMQLIEDFSTHVSIDCSPHKTVKKTANEFPCLPKQV |
| | AWILATSKVFMYPELLPVCSLKAKNPQDKIVFTKAEDNLLALGLKH |
| | FEGTEFPNPLISKYLLTCKTAHQLTVRIKNLNMNRAPDNIIKFYKKT |
| | KQLPVLGKCCEEIQPHQWKPPIEREEHRLPFWLKASLPSIQEELR |
| | HMADGAREVGNMTGTTEINSDRSLEKDNLELGSESRYPLLLPKG |
| | VVLKLKPVATRSPRKAWRQKRSSVLKPLLIQPSPSLQPSFNPGKT |
| | PARSTHSEAPPSKMVLRIPHPIQPATVLQTVPGVPPLGVSGGESF |
| | ESPAALPAVPPEARTSFPLSESQTLLSSAPVPKVMLPSLAPSKFR |
| | KPYVRRRPSKRRGVKASPCMKPAPVIHHPASVIFTVPATTVKIVS |
| | LGGGCNMIQPVNAAVAQSPQTIPITTLLVNPTSFPCPLNQSLVAS |
| | SVSPLIVSGNSVNLPIPSTPEDKAHVNVDIACAVADGENAFQGLE |
| | PKLEPQELSPLSATVFPKVEHSPGPPLADAECQEGLSENSACRW |
| | TVVKTEEGRQALEPLPQGIQESLNNPTPGDLEEIVKMEPEEAREE |
| | ISGSPERDICDDIKVEHAVELDTGAPSEELSSAGEVTKQTVLQKE |
| | EGRSQPTKTPSSSQEPPDEGTSGTDVNKGSSKNALSSMDPEVR |
| | LSSPPGKPEDSSSVDGQSVGTPVGPETGGEKNGPEEEEEEDFD |
| | DLTQDEEDEMSSASEESVLSVPELQVRAGEYSQVFRGLSNMYH |
| | LLICHLLACCTMDSPKIICI |
| SEQ ID NO 4: | MLPCKKRRTTVTESLQHKGNQEENNVDLESAVKPESDQVKDLS |
| Amino acid (AA) sequence | SVSLSWDPSHGRVAGFEVQSLQDAGNQLGMEDTSLSSGMLTQ |
| of human GON4L isoform C | NTNVPILEGVDVAISQGITLPSLESFHPLNIHIGKGKLHATGSKRGK |
| GenBank: AAR01261.1 | KMTLRPGPVTQEDRCDHLTLKEPFSGEPSEEVKEEGGKPQMNS |
| | EGEIPSLPSGSQSAKPVSQPRKSTQPDVCASPQEKPLRTLFHQP |
| | EEEIEDGGLFIPMEEQDNEESEKRRKKKKGTKRKRDGRGQEGTL |
| | AYDLKLDDMLDRTLEDGAKQHNLTAVNVRNILHEVITNEHVVAM |
| | MKAAISETEDMPMFEPKMTRSKLKEVVEKGVVIPTWNISPIKKAN |
| | EIKPPQFVDIHLEEDDSSDEEYQPDDEEEDETAEESLLESDVEST |
| | ASSPRGAKKSRLRQSSEMTETDEESGILSEAEKVTTPAIRHISAE |
| | VVPMGPPPPPKPKQTRDSTFMEKLHAVDEELASSPVCMDSFQP |
| | MDDSLIAFRTRSKMPLKDVPLGQLEAELQAPDITPDMYDPNTAD |
| | DEDWKMWLGGLMNDDVGNEDEADDDDDPEYNFLEDLDEPDTE |
| | DFRTDRAVRITKKEVNELMEELFETFQDEMGFSNMEDDGPEEEE |
| | CVAEPRPNFNTPQALRFEEPLANLLNEQHRTVKELFEQLKMKKS |
| | SAKQLQEVEKVKPQSEKVHQTLILDPAQRKRLQQQMQQHVQLLT |
| | QIHLLATCNPNLNPEATTTRIFLKELGTFAQSSIALHHQYNPKFQT |
| | LFQPCNLMGAMQLIEDFSTHVSIDCSPHKTVKKTANEFPCLPKQV |
| | AWILATSKVFMYPELLPVCSLKAKNPQDKIVFTKAEDNLLALGLKH |
| | FEGTEFPNPLISKYLLTCKTAHQLTVRIKNLNMNRAPDNIIKFYKKT |
| | KQLPVLGKCCEEIQPHQWKPPIEREEHRLPFWLKASLPSIQEELR |
| | HMADGAREVGNMTGTTEINSDRSLEKDNLELGSESRYPLLLPKG |
| | VVLKLKPVATRFPRKAWRQKRSSVLKPLLIQPSPSLQPSFNPGKT |
| | PARSTHSEAPPSKMVLRIPHPIQPATVLQTVPGVPPLGVSGGESF |
| | ESPAALPAVPPEARTSFPLSESQTLLSSAPVPKVMLPSLAPSKFR |
| | KPYVRRRPSKRRGVKASPCMKPAPVIHHPASVIFTVPATTVKIVS |
| | LGGGCNMIQPVNAAVAQSPQTIPITTLLVNPTSFPCPLNQSLVAS |
| | SVSPLIVSGNSVNLPIPSTPEDKAHVNVDIACAVADGENAFQGLE |
| | PKLEPQELSPLSATVFPKVEHSPGPPLADAECQEGLSENSACRW |
| | TVVKTEEGRQALEPLPQGIQESLNNPTPGDLEEIVKMEPEEAREE |
| | ISGSPERDICDDIKVEHAVELDTGAPSEELSSAGEVTKQTVLQKE |
| | EERSQPTKTPSSSQEPPDEGTSGTDVNKGSSKNALSSMDPEVR |
| | LSSPPGKPEDSSSVDGQSVGTPVGPETGGEKNGPEEEEEEDFD |
| | DLTQDEEDEMSSASEESVLSVPELQETMEKLTWLASERRMSQE |
| | GESEEENSQEENSEPEEEEEEEAEGMESLQKEDEMTDEAVGDS |
| | AEKPPTFASPETAPEVETSRTPPGESIKAAGKGRNNHRARNKRG |
| | SRARASKDTSKLLLLYDEDILERDPLREQKDLAFAQAYLTRVREA |
| | LQHIPGKYEDFLQVIYEFESSTQRRTAVDLYKSLQILLQDWPQLLK |
| | DFAAFLLPEQALACGLFEEQQAFEKSRKFLRQLEICFAENPSHHQ |
| | KIIKVLQGCADCLPQEITELKTQMWQLLKGHDHLQDEFSIFFDHLR |
| | PAASRMGDFEEINWTEEKEYEFDGFEEVALPDVEEEEEPPKIPTA |
| | SKNKRKKEIGVQNHDKETEWPDGAKDCACSCHEGGPDSKLKKS |
| | KRRSCSHCSSKVCDSKSYKSKEPHELVGSSPHREASPMPGAKE |
| | AGQGKDMMEEEAPEERESTEATQSRTVRTTRKGEMPVSAGLAV |
| | GSTLPSPREVTVTERLLLDGPPPHSPETPQFPPTTGAVLYTVKRN |
| | QVGPEVRSCPKASPRLQKEREGQKAVSESEALMLVWDASETEK |
| | LPGTVEPPASFLSPVSSKTRDAGRRHVSGKPDTQERWLPSSRA |
| | RVKTRDRTCPVHESPSGIDTSETSPKAPRGGLAKDSGTQAKGPE |
| | GEQQPKAAEATVCANNSKVSSTGEKVVLWTREADRVILTMCQE |
| | QGAQPQTFNIISQQLGNKTPAEVSHRFRELMQLFHTACEASSED |
| | EDDATSTSNADQLSDHGDLLSEEELDE |
| SEQ ID NO 5: | MTKSYSESGLMGEPQPQGPPSWTDECLSSQDEEHEADKKEDD |
| Amino acid (AA) sequence | LEAMNAEEDSLRNGGEEEDEDEDLEEEEEEEEEDDDQKPKRRG |
| of human NEUROD1 | PKKKKMTKARLERFKLRRMKANARERNRMHGLNAALDNLRKVV |
| GenBank: BAJ84018.1 | PCYSKTQKLSKIETLRLAKNYIWALSEILRSGKSPDLVSFVQTLCK |
| | GLSQPTTNLVAGCLQLNPRTFLPEQNQDMPPHLPTASASFPVHP |
| | YSYQSPGLPSPPYGTMDSSHVFHVKPPPHAYSAALEPFFESPLT |
| | DCTSPSFDGPLSPPLSINGNFSFKHEPSAEFEKNYAFTMHYPAAT |
| | LAGAQSHGSIFSGTAAPRCEIPIDNIMSFDSHSHHERVMSAQLNAI |
| | FHD |
| SEQ ID NO 6: | MMSYLKQPPYAVNGLSLTTSGMDLLHPSVGYPGPWASCPAATP |
| Amino acid (AA) sequence | RKQRRERTTFTRAQLDVLEALFAKTRYPDIFMREEVALKINLPES |
| of human OTX2 Isoform A | RVQVWFKNRRAKCRQQQQQQQNGGQNKVRPAKKKTSPAREV |
| NCBI Reference | SSESGTSGQFTPPSSTSVPTIASSSAPVSIWSPASISPLSDPLSTS |
| Sequence: NP_068374.1 | SSCMQRSYPMTYTQASGYSQGYAGSTSYFGGMDCGSYLTPMH |
| | HQLPGPGATLSPMGTNAVTSHLNQSPASLSTQGYGASSLGFNS |
| | TTDCLDYKDQTASWKLNFNADCLDYKDQTSSWKFQVL |
| SEQ ID NO 7: | MMSYLKQPPYAVNGLSLTTSGMDLLHPSVGYPATPRKQRRERT |
| Amino acid (AA) sequence | TFTRAQLDVLEALFAKTRYPDIFMREEVALKINLPESRVQVWFKN |
| of human OTX2 Isoform B | RRAKCRQQQQQQQNGGQNKVRPAKKKTSPAREVSSESGTSGQ |
| NCBI Reference | FTPPSSTSVPTIASSSAPVSIWSPASISPLSDPLSTSSSCMQRSYP |
| Sequence: | MTYTQASGYSQGYAGSTSYFGGMDCGSYLTPMHHQLPGPGAT |
| NP_001257453.1 | LSPMGTNAVTSHLNQSPASLSTQGYGASSLGFNSTTDCLDYKDQ |
| | TASWKLNFNADCLDYKDQTSSWKFQVL |

In the context of the present invention, the provision of GON4L isoform B according to SEQ ID NO 3 and/or OTX2 isoform A according to SEQ ID NO 6 is particularly advantageous.

The invention further relates to functionally analogous sequences of the respective TF. Protein modifications to the TF of the present invention, which may occur through substitutions in amino acid sequence, and nucleic acid sequences encoding such molecules, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, lie and Leu; the non-polar aromatic amino acids Phe, Trp and Tyr; the neutral polar amino acids Ser, Thr, Cys, Gin, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

As explained herein, in the context of the invention the one or more TF may be provided at the protein level or in the form of a nucleic acid encoding a TF.

Nucleic acid sequences of the invention include the nucleic acid sequences encoding GON4L, NEUROD1 and OTX2 protein sequences according to Table 1 and functionally analogous sequences. Preferred nucleic acid sequence encoding GON4L, NEUROD1 and OTX2 protein are listed under Table 2.

The TF of the invention may include proteins tags that allow easy identification of the provided TF in the cell through standard techniques, for example by using antibodies directed against the protein tag. A preferred protein-tag that can be encoded by a nucleic acid sequence of the invention is a V5-tag. Alternative tags may be used instead of a V5-tag. Such alternatives are well known in the art and can be selected by a skilled person.

TABLE 2
Nucleic acid sequences of preferred TF of the invention.

| | |
|---|---|
| SEQ ID NO 8: | ATGTTGCCCTGTAAGAAGAGAAGAACTACAGTGACAGAGTCC |
| sequence of human | CTACAGCATAAAGGCAATCAAGAGGAAAACAACGTAGACCTA |
| Coding nucleic acid | GAATCAGCCGTTAAACCAGAATCTGACCAGGTTAAGGACTTGA |
| GON4L isoform B | CTGGCTTCGAAGTACAGTCTTTGCAGGATGCAGGAAATCAGC |
| | GTTCGGTGTCACTATCCTGGGATCCAAGTCATGGCAGAGTAG |
| | TTGGTATGGAGGATACATCTCTGAGCTCTGGAATGCTCACCCA |
| | GAACACAAATGTACCAATTCTAGAAGGTGTTGATGTGGCCATC |
| | TCTCAGGGAATCACCCTACCTTCCTTGGAGTCTTTTCACCCCC |
| | TTAATATACACATTGGTAAAGGAAAACTCCACGCTACTGGCTC |
| | AAAGAGAGGGAAAAAAATGACACTCAGGCCTGGGCCAGTTAC |
| | CCAAGAAGACAGATGTGATCATCTTACCCTAAAGGAGCCTTTT |
| | TCAGGAGAGCCTAGTGAAGAAGTCAAGGAAGAAGGAGGGAAA |
| | CCTCAAATGAATTCTGAAGGGGAGATACCTTCCCTGCCATCAG |
| | GCAGCCAATCTGCAAAACCAGTAAGCCAGCCCAGGAAATCAA |
| | CCCAGCCAGATGTTTGTGCCTCTCCTCAAGAAAAGCCACTCA |
| | GGACTCTGTTTCACCAACCTGAGGAAGAGATAGAAGATGGTG |
| | GACTCTTCATTCCAATGGAAGAACAAGACAATGAAGAAAGTGA |
| | GAAAAGGAGAAAAAAGAAAAAGGGTACCAAGAGGAAACGAGA |
| | TGGAAGGGGTCAAGAAGGGACCTTGGCATATGACCTGAAACT |
| | GGATGACATGCTTGACCGTACCTTGGAGGATGGTGCCAAGCA |
| | GCACAATCTAACAGCAGTCAATGTCCGAAACATCCTTCATGAA |
| | GTAATCACAAATGAACACGTGGTAGCTATGATGAAAGCAGCCA |
| | TCAGTGAGACGGAAGATATGCCAATGTTTGAGCCTAAAATGAC |
| | ACGCTCTAAACTGAAGGAAGTAGTGGAAAAAGGAGTGGTAATT |
| | CCAACATGGAATATTTCACCAATTAAGAAGGCCAATGAAATTA |
| | AGCCTCCTCAGTTTGTGGATATCCACCTTGAAGAAGATGATTC |
| | CTCAGATGAAGAATACCAGCCGGATGATGAAGAAGAAGATGA |
| | AACTGCTGAAGAGAGCTTATTGGAAAGTGATGTTGAAAGCACT |
| | GCTTCATCTCCACGTGGGGCAAAGAAATCCAGATTGAGGCAG |
| | TCTTCTGAGATGACTGAAACAGATGAGGAGAGTGGCATATTAT |
| | CAGAGGCTGAGAAAGTCACCACACCAGCCATCAGGCACATCA |
| | GTGCTGAGGTAGTGCCCATGGGGCCCCCGCCCCCTCCAAAG |
| | CCGAAACAGACCAGAGATAGTACTTTCATGGAGAAGTTACATG |
| | CGGTAGATGAGGAGCTGGCTTCCAGTCCAGTCTGCATGGATT |
| | CTTTCCAGCCCATGGATGACAGTCTCATTGCATTTCGAACGCG |
| | TTCTAAGATGCCCCTGAAAGATGTTCCCCTGGGCCAATTAGAG |
| | GCAGAGCTCCAAGCTCCAGACATCACTCCAGATATGTATGAC |
| | CCCAATACGGCAGATGATGAGGACTGGAAGATGTGGCTGGG |
| | GGGACTTATGAATGATGATGTGGGGAATGAAGATGAAGCAGA |
| | TGATGATGATGATCCAGAATATAATTTCCTGGAAGACCTCGAT |
| | GAACCAGACACAGAGGATTTCCGGACTGACCGGGCAGTGAGA |
| | ATCACCAAAAAGGAAGTAAATGAGCTGATGGAAGAGCTGTTTG |
| | AAACTTTCCAAGATGAGATGGGATTCTCCAACATGGAAGATGA |
| | TGGCCCAGAAGAGGAGGAGTGTGTAGCTGAGCCTCGTCCTAA |
| | CTTTAACACCCCTCAAGCTCTACGGTTTGAGGAACCACTGGCC |
| | AACCTGTTAAATGAACAACATCGGACAGTGAAGGAGCTATTTG |
| | AACAGCTGAAGATGAAGAAATCTTCAGCCAAACAGCTGCAGG |
| | AAGTAGAGAAGGTTAAACCCCAGAGTGAGAAAGTTCATCAGA |
| | CTCTGATTCTGGACCCAGCACAGAGGAAGAGACTCCAGCAGC |
| | AGATGCAGCAGCACGTTCAGCTCTTGACCCAAATCCACCTTCT |
| | TGCCACCTGCAACCCCAACCTCAATCCGGAGGCCACTACCAC |
| | CAGGATATTTCTTAAAGAGCTGGGAACCTTTGCTCAAAGCTCC |
| | ATCGCCCTTCACCATCAGTACAACCCCAAGTTTCAGACCCTGT |
| | TCCAACCCTGTAACTTGATGGGAGCTATGCAGCTGATTGAAGA |
| | CTTCAGCACACATGTCAGCATTGACTGCAGCCCTCATAAAACT |
| | GTCAAGAAGACTGCGAATGAATTTCCCTGTTTGCCAAAGCAAG |
| | TGGCTTGGATTCTGGCCACAAGCAAGGTTTTCATGTATCCAGA |
| | GTTACTTCCAGTGTGTTCCCTGAAGGCAAAGAATCCCCAGGAT |
| | AAGATCGTCTTCACCAAGGCTGAGGACAATTTGTTAGCTTTAG |
| | GACTGAAGCATTTTGAAGGAACTGAGTTTCCTAATCCTCTAAT |
| | CAGCAAGTACCTTCTAACCTGCAAAACTGCCCACCAACTGACA |
| | GTGAGAATCAAGAACCTCAACATGAACAGAGCTCCTGACAACA |
| | TCATTAAATTTTATAAGAAGACCAAACAGCTGCCAGTCCTAGG |
| | AAAATGCTGTGAAGAGATCCAGCCACATCAGTGGAAGCCACC |
| | TATAGAGAGAGAAGAACACCGGCTCCCATTCTGGTTAAAGGC |
| | CAGTCTGCCATCCATCCAGGAAGAACTGCGGCACATGGCTGA |
| | TGGTGCTAGAGAGGTAGGAAATATGACTGGAACCACTGAGAT |
| | CAACTCAGATCGAAGCCTAGAAAAAGACAATTTGGAGTTGGG |
| | GAGTGAATCTCGGTACCCACTGCTATTGCCTAAGGGTGTAGT |
| | CCTGAAACTGAAGCCAGTTGCCACCCGTTTCCCCAGGAAGGC |
| | TTGGAGACAGAAGCGTTCATCAGTCCTGAAGCCCCTCCTTATC |
| | CAACCCAGCCCCTCTCTCCAGCCCAGCTTCAACCCTGGGAAA |
| | ACACCAGCCCGATCAACTCATTCAGAAGCCCCTCCGAGCAAA |
| | ATGGTGCTCCGGATTCCTCACCCAATACAGCCAGCCACTGTTT |
| | TACAGACAGTTCCAGGTGTCCCTCCACTGGGGGTCAGTGGAG |
| | GTGAGAGTTTTGAGTCTCCTGCAGCACTGCCTGCTGTGCCCC |
| | CTGAGGCCAGGACAAGCTTCCCTCTGTCTGAGTCCCAGACTT |
| | TGCTCTCTTCTGCCCCTGTGCCCAAGGTAATGCTGCCCTCCCT |
| | TGCCCCTTCTAAGTTTCGAAAGCCATATGTGAGACGGAGACC |
| | CTCAAAGAGAAGAGGAGTCAAGGCCTCTCCCTGTATGAAACC |
| | TGCCCCTGTTATCCACCACCCTGCATCTGTTATCTTCACTGTT |
| | CCTGCTACCACTGTGAAGATTGTGAGCCTTGGCGGTGGCTGT |
| | AACATGATCCAGCCTGTCAATGCGGCTGTGGCCCAGAGTCCC |
| | CAGACTATTCCCATCACTACCCTCTTGGTTAACCCTACTTCCTT |
| | CCCCTGTCCATTGAACCAGTCCCTTGTGGCCTCCTCTGTCTCA |
| | CCCTTAATTGTTTCTGGCAATTCTGTGAATCTTCCTATACCATC |
| | CACCCCTGAAGATAAGGCCCACGTGAATGTGGACATTGCTTG |
| | TGCTGTGGCTGATGGGGAAAATGCCTTTCAGGGCCTAGAACC |
| | CAAATTAGAGCCCCAGGAACTATCTCCTCTCTCTGCTACTGTT |
| | TTCCCGAAAGTGGAACATAGCCCAGGGCCTCCACTAGCAGAT |
| | GCAGAGTGCCAAGAAGGATTGTCAGAGAATAGTGCCTGTCGC |
| | TGGACCGTTGTGAAAACAGAGGAGGGGAGGCAAGCTCTGGA |
| | GCCGCTCCCTCAGGGCATCCAGGAGTCTCTAAACAACCCTAC |
| | CCCTGGGGATTTAGAGGAAATTGTCAAGATGGAACCTGAAGA |
| | AGCTAGAGAGGAAATCAGTGGATCCCCTGAGCGTGATATTTG |
| | TGATGACATCAAAGTGGAACATGCTGTGGAATTGGACACTGGT |
| | GCCCCAAGCGAGGAGTTGAGCAGTGCTGGAGAAGTAACGAAA |
| | CAGACAGTCTTACAGAAGGAAGAGGAGAGGAGTCAGCCAACT |
| | AAAACCCCTTCATCTTCTCAAGAGCCCCCTGATGAAGGAACCT |
| | CAGGGACAGATGTGAACAAAGGATCATCAAAGAATGCTTTGTC |
| | CTCAATGGATCCTGAAGTGAGGCTTAGTAGCCCCCCAGGGAA |
| | GCCAGAAGATTCATCCAGTGTTGATGGTCAGTCAGTGGGGAC |
| | TCCAGTTGGGCCAGAAACTGGAGGAGAGAAGAATGGGCCAG |
| | AAGAAGAGGAAGAAGAGGACTTTGATGACCTCACCCAAGATG |
| | AGGAAGATGAAATGTCATCAGCTTCTGAGGAATCTGTGCTTTC |
| | TGTCCCAGAACTCCAGGTGAGAGCTGGAGAATATTCTCAAGTA |
| | TTTCGTGGACTCAGTAATATGTATCACTTATTGATATGCCACCT |
| | GCTTGCTTGCTGCACTATGGATAGTCCTAAAATCATTTGTATT |
| SEQ ID NO 9: | ATGTTGCCCTGTAAGAAGAGAAGAACTACAGTGACAGAGTCC |
| Coding nucleic acid | CTACAGCATAAAGGCAATCAAGAGGAAAACAACGTAGACCTA |
| sequence of human | GAATCAGCCGTTAAACCAGAATCTGACCAGGTTAAGGACTTGA |
| GON4L isoform B V5 | GTTCGGTGTCACTATCCTGGGATCCAAGTCATGGCAGAGTAG |
| (comprising a V5-tag at | CTGGCTTCGAAGTACAGTCTTTGCAGGATGCAGGAAATCAGC |
| the 3′ end) | TTGGTATGGAGGATACATCTCTGAGCTCTGGAATGCTCACCCA |
| | GAACACAAATGTACCAATTCTAGAAGGTGTTGATGTGGCCATC |
| | TCTCAGGGAATCACCCTACCTTCCTTGGAGTCTTTTCACCCCC |
| | TTAATATACACATTGGTAAAGGAAAACTCCACGCTACTGGCTC |
| | AAAGAGAGGGAAAAAAATGACACTCAGGCCTGGGCCAGTTAC |
| | CCAAGAAGACAGATGTGATCATCTTACCCTAAAGGAGCCTTTT |
| | TCAGGAGAGCCTAGTGAAGAAGTCAAGGAAGAAGGAGGGAAA |
| | CCTCAAATGAATTCTGAAGGGGAGATACCTTCCCTGCCATCAG |
| | GCAGCCAATCTGCAAAACCAGTAAGCCAGCCCAGGAAATCAA |
| | CCCAGCCAGATGTTTGTGCCTCTCCTCAAGAAAAGCCACTCA |
| | GGACTCTGTTTCACCAACCTGAGGAAGAGATAGAAGATGGTG |
| | GACTCTTCATTCCAATGGAAGAACAAGACAATGAAGAAAGTGA |
| | GAAAAGGAGAAAAAAGAAAAAGGGTACCAAGAGGAAACGAGA |
| | TGGAAGGGGTCAAGAAGGGACCTTGGCATATGACCTGAAACT |
| | GGATGACATGCTTGACCGTACCTTGGAGGATGGTGCCAAGCA |
| | GCACAATCTAACAGCAGTCAATGTCCGAAACATCCTTCATGAA |
| | GTAATCACAAATGAACACGTGGTAGCTATGATGAAAGCAGCCA |
| | TCAGTGAGACGGAAGATATGCCAATGTTTGAGCCTAAAATGAC |
| | ACGCTCTAAACTGAAGGAAGTAGTGGAAAAAGGAGTGGTAATT |
| | CCAACATGGAATATTTCACCAATTAAGAAGGCCAATGAAATTA |
| | AGCCTCCTCAGTTTGTGGATATCCACCTTGAAGAAGATGATTC |
| | CTCAGATGAAGAATACCAGCCGGATGATGAAGAAGAAGATGA |
| | AACTGCTGAAGAGAGCTTATTGGAAAGTGATGTTGAAAGCACT |
| | GCTTCATCTCCACGTGGGGCAAAGAAATCCAGATTGAGGCAG |
| | TCTTCTGAGATGACTGAAACAGATGAGGAGAGTGGCATATTAT |
| | CAGAGGCTGAGAAAGTCACCACACCAGCCATCAGGCACATCA |
| | GTGCTGAGGTAGTGCCCATGGGGCCCCCGCCCCCTCCAAAG |
| | CCGAAACAGACCAGAGATAGTACTTTCATGGAGAAGTTACATG |
| | CGGTAGATGAGGAGCTGGCTTCCAGTCCAGTCTGCATGGATT |
| | CTTTCCAGCCCATGGATGACAGTCTCATTGCATTTCGAACGCG |
| | TTCTAAGATGCCCCTGAAAGATGTTCCCCTGGGCCAATTAGAG |
| | GCAGAGCTCCAAGCTCCAGACATCACTCCAGATATGTATGAC |
| | CCCAATACGGCAGATGATGAGGACTGGAAGATGTGGCTGGG |
| | GGGACTTATGAATGATGATGTGGGGAATGAAGATGAAGCAGA |
| | TGATGATGATGATCCAGAATATAATTTCCTGGAAGACCTCGAT |
| | GAACCAGACACAGAGGATTTCCGGACTGACCGGGCAGTGAGA |
| | ATCACCAAAAAGGAAGTAAATGAGCTGATGGAAGAGCTGTTTG |
| | AAACTTTCCAAGATGAGATGGGATTCTCCAACATGGAAGATGA |
| | TGGCCCAGAAGAGGAGGAGTGTGTAGCTGAGCCTCGTCCTAA |
| | CTTTAACACCCCTCAAGCTCTACGGTTTGAGGAACCACTGGCC |
| | AACCTGTTAAATGAACAACATCGGACAGTGAAGGAGCTATTTG |
| | AACAGCTGAAGATGAAGAAATCTTCAGCCAAACAGCTGCAGG |
| | AAGTAGAGAAGGTTAAACCCCAGAGTGAGAAAGTTCATCAGA |
| | CTCTGATTCTGGACCCAGCACAGAGGAAGAGACTCCAGCAGC |
| | AGATGCAGCAGCACGTTCAGCTCTTGACCCAAATCCACCTTCT |
| | TGCCACCTGCAACCCCAACCTCAATCCGGAGGCCACTACCAC |
| | CAGGATATTTCTTAAAGAGCTGGGAACCTTTGCTCAAAGCTCC |
| | ATCGCCCTTCACCATCAGTACAACCCCAAGTTTCAGACCCTGT |
| | TCCAACCCTGTAACTTGATGGGAGCTATGCAGCTGATTGAAGA |
| | CTTCAGCACACATGTCAGCATTGACTGCAGCCCTCATAAAACT |
| | GTCAAGAAGACTGCGAATGAATTTCCCTGTTTGCCAAAGCAAG |
| | TGGCTTGGATTCTGGCCACAAGCAAGGTTTTCATGTATCCAGA |
| | GTTACTTCCAGTGTGTTCCCTGAAGGCAAAGAATCCCCAGGAT |
| | AAGATCGTCTTCACCAAGGCTGAGGACAATTTGTTAGCTTTAG |
| | GACTGAAGCATTTTGAAGGAACTGAGTTTCCTAATCCTCTAAT |
| | CAGCAAGTACCTTCTAACCTGCAAAACTGCCCACCAACTGACA |
| | GTGAGAATCAAGAACCTCAACATGAACAGAGCTCCTGACAACA |
| | TCATTAAATTTTATAAGAAGACCAAACAGCTGCCAGTCCTAGG |
| | AAAATGCTGTGAAGAGATCCAGCCACATCAGTGGAAGCCACC |
| | TATAGAGAGAGAAGAACACCGGCTCCCATTCTGGTTAAAGGC |
| | CAGTCTGCCATCCATCCAGGAAGAACTGCGGCACATGGCTGA |
| | TGGTGCTAGAGAGGTAGGAAATATGACTGGAACCACTGAGAT |
| | CAACTCAGATCGAAGCCTAGAAAAAGACAATTTGGAGTTGGG |
| | GAGTGAATCTCGGTACCCACTGCTATTGCCTAAGGGTGTAGT |
| | CCTGAAACTGAAGCCAGTTGCCACCCGTTTCCCCAGGAAGGC |
| | TTGGAGACAGAAGCGTTCATCAGTCCTGAAGCCCCTCCTTATC |
| | CAACCCAGCCCCTCTCTCCAGCCCAGCTTCAACCCTGGGAAA |
| | ACACCAGCCCGATCAACTCATTCAGAAGCCCCTCCGAGCAAA |
| | ATGGTGCTCCGGATTCCTCACCCAATACAGCCAGCCACTGTTT |
| | TACAGACAGTTCCAGGTGTCCCTCCACTGGGGGTCAGTGGAG |
| | GTGAGAGTTTTGAGTCTCCTGCAGCACTGCCTGCTGTGCCCC |
| | CTGAGGCCAGGACAAGCTTCCCTCTGTCTGAGTCCCAGACTT |
| | TGCTCTCTTCTGCCCCTGTGCCCAAGGTAATGCTGCCCTCCCT |
| | TGCCCCTTCTAAGTTTCGAAAGCCATATGTGAGACGGAGACC |
| | CTCAAAGAGAAGAGGAGTCAAGGCCTCTCCCTGTATGAAACC |
| | TGCCCCTGTTATCCACCACCCTGCATCTGTTATCTTCACTGTT |
| | CCTGCTACCACTGTGAAGATTGTGAGCCTTGGCGGTGGCTGT |
| | AACATGATCCAGCCTGTCAATGCGGCTGTGGCCCAGAGTCCC |
| | CAGACTATTCCCATCACTACCCTCTTGGTTAACCCTACTTCCTT |
| | CCCCTGTCCATTGAACCAGTCCCTTGTGGCCTCCTCTGTCTCA |
| | CCCTTAATTGTTTCTGGCAATTCTGTGAATCTTCCTATACCATC |
| | CACCCCTGAAGATAAGGCCCACGTGAATGTGGACATTGCTTG |
| | TGCTGTGGCTGATGGGGAAAATGCCTTTCAGGGCCTAGAACC |
| | CAAATTAGAGCCCCAGGAACTATCTCCTCTCTCTGCTACTGTT |
| | TTCCCGAAAGTGGAACATAGCCCAGGGCCTCCACTAGCAGAT |
| | GCAGAGTGCCAAGAAGGATTGTCAGAGAATAGTGCCTGTCGC |
| | TGGACCGTTGTGAAAACAGAGGAGGGGAGGCAAGCTCTGGA |
| | GCCGCTCCCTCAGGGCATCCAGGAGTCTCTAAACAACCCTAC |
| | CCCTGGGGATTTAGAGGAAATTGTCAAGATGGAACCTGAAGA |
| | AGCTAGAGAGGAAATCAGTGGATCCCCTGAGCGTGATATTTG |
| | TGATGACATCAAAGTGGAACATGCTGTGGAATTGGACACTGGT |
| | GCCCCAAGCGAGGAGTTGAGCAGTGCTGGAGAAGTAACGAAA |
| | CAGACAGTCTTACAGAAGGAAGAGGAGAGGAGTCAGCCAACT |
| | AAAACCCCTTCATCTTCTCAAGAGCCCCCTGATGAAGGAACCT |
| | CAGGGACAGATGTGAACAAAGGATCATCAAAGAATGCTTTGTC |
| | CTCAATGGATCCTGAAGTGAGGCTTAGTAGCCCCCCAGGGAA |
| | GCCAGAAGATTCATCCAGTGTTGATGGTCAGTCAGTGGGGAC |
| | TCCAGTTGGGCCAGAAACTGGAGGAGAGAAGAATGGGCCAG |
| | AAGAAGAGGAAGAAGAGGACTTTGATGACCTCACCCAAGATG |
| | AGGAAGATGAAATGTCATCAGCTTCTGAGGAATCTGTGCTTTC |
| | TGTCCCAGAACTCCAGGTGAGAGCTGGAGAATATTCTCAAGTA |
| | TTTCGTGGACTCAGTAATATGTATCACTTATTGATATGCCACCT |
| | GCTTGCTTGCTGCACTATGGATAGTCCTAAAATCATTTGTATTC |
| | TCGAGGGTAAGCCTATCCCTAACCCTCTCCTCGGTCTCGATTC |
| | TACGTAATGA |
| SEQ ID NO 10: | ATGACCAAATCGTACAGCGAGAGTGGGCTGATGGGCGAGCCT |
| Coding nucleic acid | CAGCCCCAAGGTCCTCCAAGCTGGACAGACGAGTGTCTCAGT |
| sequence of human | TCTCAGGACGAGGAGCACGAGGCAGACAAGAAGGAGGACGA |
| NEUROD1 | CCTCGAAGCCATGAACGCAGAGGAGGACTCACTGAGGAACG |
| | GGGGAGAGGAGGAGGACGAAGATGAGGACCTGGAAGAGGAG |
| | GAAGAAGAGGAAGAGGAGGATGACGATCAAAAGCCCAAGAGA |
| | CGCGGCCCCAAAAAGAAGAAGATGACTAAGGCTCGCCTGGAG |
| | CGTTTTAAATTGAGACGCATGAAGGCTAACGCCCGGGAGCGG |
| | AACCGCATGCACGGACTGAACGCGGCGCTAGACAACCTGCG |
| | CAAGGTGGTGCCTTGCTATTCTAAGACGCAGAAGCTGTCCAA |
| | AATCGAGACTCTGCGCTTGGCCAAGAACTACATCTGGGCTCT |
| | GTCGGAGATCCTGCGCTCAGGCAAAAGCCCAGACCTGGTCTC |
| | CTTCGTTCAGACGCTTTGCAAGGGCTTATCCCAACCCACCACC |
| | AACCTGGTTGCGGGCTGCCTGCAACTCAATCCTCGGACTTTT |
| | CTGCCTGAGCAGAACCAGGACATGCCCCCCCACCTGCCGAC |
| | GGCCAGCGCTTCCTTCCCTGTACACCCCTACTCCTACCAGTC |
| | GCCTGGGCTGCCCAGTCCGCCTTACGGTACCATGGACAGCTC |
| | CCATGTCTTCCACGTTAAGCCTCCGCCGCACGCCTACAGCGC |
| | AGCGCTGGAGCCCTTCTTTGAAAGCCCTCTGACTGATTGCAC |
| | CAGCCCTTCCTTTGATGGACCCCTCAGCCCGCCGCTCAGCAT |
| | CAATGGCAACTTCTCTTTCAAACACGAACCGTCCGCCGAGTTT |
| | GAGAAAAATTATGCCTTTACCATGCACTATCCTGCAGCGACAC |
| | TGGCAGGGGCCCAAAGCCACGGATCAATCTTCTCAGGCACCG |
| | CTGCCCCTCGCTGCGAGATCCCCATAGACAATATTATGTCCTT |
| | CGATAGCCATTCACATCATGAGCGAGTCATGAGTGCCCAGCT |
| | CAATGCCATATTTCATGAT |
| SEQ ID NO 11: | ATGACCAAATCGTACAGCGAGAGTGGGCTGATGGGCGAGCCT |
| Coding nucleic acid | CAGCCCCAAGGTCCTCCAAGCTGGACAGACGAGTGTCTCAGT |
| sequence of human | TCTCAGGACGAGGAGCACGAGGCAGACAAGAAGGAGGACGA |
| NEUROD1 V5 (comprising | CCTCGAAGCCATGAACGCAGAGGAGGACTCACTGAGGAACG |
| a V5-tag at the 3′ end) | GGGGAGAGGAGGAGGACGAAGATGAGGACCTGGAAGAGGAG |
| | GAAGAAGAGGAAGAGGAGGATGACGATCAAAAGCCCAAGAGA |
| | CGCGGCCCCAAAAAGAAGAAGATGACTAAGGCTCGCCTGGAG |
| | CGTTTTAAATTGAGACGCATGAAGGCTAACGCCCGGGAGCGG |
| | AACCGCATGCACGGACTGAACGCGGCGCTAGACAACCTGCG |
| | CAAGGTGGTGCCTTGCTATTCTAAGACGCAGAAGCTGTCCAA |
| | AATCGAGACTCTGCGCTTGGCCAAGAACTACATCTGGGCTCT |
| | GTCGGAGATCCTGCGCTCAGGCAAAAGCCCAGACCTGGTCTC |
| | CTTCGTTCAGACGCTTTGCAAGGGCTTATCCCAACCCACCACC |
| | AACCTGGTTGCGGGCTGCCTGCAACTCAATCCTCGGACTTTT |
| | CTGCCTGAGCAGAACCAGGACATGCCCCCCCACCTGCCGAC |
| | GGCCAGCGCTTCCTTCCCTGTACACCCCTACTCCTACCAGTC |
| | GCCTGGGCTGCCCAGTCCGCCTTACGGTACCATGGACAGCTC |
| | CCATGTCTTCCACGTTAAGCCTCCGCCGCACGCCTACAGCGC |
| | AGCGCTGGAGCCCTTCTTTGAAAGCCCTCTGACTGATTGCAC |
| | CAGCCCTTCCTTTGATGGACCCCTCAGCCCGCCGCTCAGCAT |
| | CAATGGCAACTTCTCTTTCAAACACGAACCGTCCGCCGAGTTT |
| | GAGAAAAATTATGCCTTTACCATGCACTATCCTGCAGCGACAC |
| | TGGCAGGGGCCCAAAGCCACGGATCAATCTTCTCAGGCACCG |
| | CTGCCCCTCGCTGCGAGATCCCCATAGACAATATTATGTCCTT |
| | CGATAGCCATTCACATCATGAGCGAGTCATGAGTGCCCAGCT |
| | CAATGCCATATTTCATGATCTCGAGGGTAAGCCTATCCCTAAC |
| | CCTCTCCTCGGTCTCGATTCTACGTAATGA |
| SEQ ID NO 12: | ATGATGTCTTATCTTAAGCAACCGCCTTACGCAGTCAATGGGC |
| Coding nucleic acid | TGAGTCTGACCACTTCGGGTATGGACTTGCTGCACCCCTCCG |
| sequence of human OTX2 | TGGGCTACCCGGGGCCCTGGGCTTCTTGTCCCGCAGCCACC |
| Isoform A | CCCCGGAAACAGCGCCGGGAGAGGACGACGTTCACTCGGGC |
| | GCAGCTAGATGTGCTGGAAGCACTGTTTGCCAAGACCCGGTA |
| | CCCAGACATCTTCATGCGAGAGGAGGTGGCACTGAAAATCAA |
| | CTTGCCCGAGTCGAGGGTGCAGGTATGGTTTAAGAATCGAAG |
| | AGCTAAGTGCCGCCAACAACAGCAACAACAGCAGAATGGAGG |
| | TCAAAACAAAGTGAGACCTGCCAAAAAGAAGACATCTCCAGCT |
| | CGGGAAGTGAGTTCAGAGAGTGGAACAAGTGGCCAATTCACT |
| | CCCCCCTCTAGCACCTCAGTCCCGACCATTGCCAGCAGCAGT |
| | GCTCCTGTGTCTATCTGGAGCCCAGCTTCCATCTCCCCACTGT |
| | CAGATCCCTTGTCCACCTCCTCTTCCTGCATGCAGAGGTCCTA |
| | TCCCATGACCTATACTCAGGCTTCAGGTTATAGTCAAGGATAT |
| | GCTGGCTCAACTTCCTACTTTGGGGGCATGGACTGTGGATCA |
| | TATTTGACCCCTATGCATCACCAGCTTCCCGGACCAGGGGCC |
| | ACACTCAGTCCCATGGGTACCAATGCAGTCACCAGCCATCTC |
| | AATCAGTCCCCAGCTTCTCTTTCCACCCAGGGATATGGAGCTT |
| | CAAGCTTGGGTTTTAACTCAACCACTGATTGCTTGGATTATAA |
| | GGACCAAACTGCCTCCTGGAAGCTTAACTTCAATGCTGACTGC |
| | TTGGATTATAAAGATCAGACATCCTCGTGGAAATTCCAGGTTT |
| | TG |
| SEQ ID NO 13: | ATGATGTCTTATCTTAAGCAACCGCCTTACGCAGTCAATGGGC |
| Coding nucleic acid | TGAGTCTGACCACTTCGGGTATGGACTTGCTGCACCCCTCCG |
| sequence of human OTX2 | TGGGCTACCCGGGGCCCTGGGCTTCTTGTCCCGCAGCCACC |
| Isoform A V5 (comprising | CCCCGGAAACAGCGCCGGGAGAGGACGACGTTCACTCGGGC |
| a V5-tag at the 3′ end) | GCAGCTAGATGTGCTGGAAGCACTGTTTGCCAAGACCCGGTA |
| | CCCAGACATCTTCATGCGAGAGGAGGTGGCACTGAAAATCAA |
| | CTTGCCCGAGTCGAGGGTGCAGGTATGGTTTAAGAATCGAAG |
| | AGCTAAGTGCCGCCAACAACAGCAACAACAGCAGAATGGAGG |
| | TCAAAACAAAGTGAGACCTGCCAAAAAGAAGACATCTCCAGCT |
| | CGGGAAGTGAGTTCAGAGAGTGGAACAAGTGGCCAATTCACT |
| | CCCCCCTCTAGCACCTCAGTCCCGACCATTGCCAGCAGCAGT |
| | GCTCCTGTGTCTATCTGGAGCCCAGCTTCCATCTCCCCACTGT |
| | CAGATCCCTTGTCCACCTCCTCTTCCTGCATGCAGAGGTCCTA |
| | TCCCATGACCTATACTCAGGCTTCAGGTTATAGTCAAGGATAT |
| | GCTGGCTCAACTTCCTACTTTGGGGGCATGGACTGTGGATCA |
| | TATTTGACCCCTATGCATCACCAGCTTCCCGGACCAGGGGCC |
| | ACACTCAGTCCCATGGGTACCAATGCAGTCACCAGCCATCTC |
| | AATCAGTCCCCAGCTTCTCTTTCCACCCAGGGATATGGAGCTT |
| | CAAGCTTGGGTTTTAACTCAACCACTGATTGCTTGGATTATAA |
| | GGACCAAACTGCCTCCTGGAAGCTTAACTTCAATGCTGACTGC |
| | TTGGATTATAAAGATCAGACATCCTCGTGGAAATTCCAGGTTT |
| | TGCTCGAGGGTAAGCCTATCCCTAACCCTCTCCTCGGTCTCG |
| | ATTCTACGTAATGA |

In another aspect, the invention encompasses the use of one or more TF, and in particular one or more nucleic acid molecules encoding GON4L and optionally NEUROD1 and OTX2, selected from the group comprising:
a) one or more nucleic acid molecules comprising a nucleotide sequence which encodes human GON4L, preferably according to SEQ ID No. 8, and optionally nucleotide sequences encoding NEUROD1, preferably according to SEQ ID No. 10, and OTX2, preferably according to SEQ ID No. 12;
b) one or more nucleic acid molecules which are complementary to the nucleotide sequences in accordance with a);
c) one or more nucleic acid molecules which undergo hybridization with the nucleotide sequences according to a) or b) under stringent conditions;
d) one or more nucleic acid molecules comprising a nucleotide sequence having sufficient sequence identity to be functionally analogous the nucleotide sequences according to a), b) or c);
e) one or more nucleic acid molecules which, as a consequence of the genetic code, are degenerated into nucleotide sequences according to a) through d); and
f) one or more nucleic acid molecules according the nucleotide sequences of a) through e) which are modified by deletions, additions, substitutions, translocations, inversions and/or insertions and functionally analogous to a nucleotide sequence according to a) through e)

Accordingly, the invention encompasses nucleic acid molecules with at least 60%, preferably 70%, more preferably 80%, especially preferably 90% sequence identity to the nucleic acid molecule encoding GON4L, and preferably NEUROD1 and OTX2.

Sequence variants of the claimed nucleic acids and/or proteins, for example defined by the provided % sequence identity, that maintain the said properties of the invention are also included in the scope of the invention. Such variants, which show alternative sequences, but maintain essentially the same properties, such as GON4L function and optionally NEUROD1 and OTX2 function, as the specific sequences provided are known as functional analogues, or as functionally analogous. Sequence identity relates to the percentage of identical nucleotides or amino acids when carrying out a sequence alignment, for example using software such as BLAST.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

In the context of the invention the term “micro-RNA” or microRNA/miRNA refers to a small non-coding RNA molecule found in plants, animals and some viruses, that functions in RNA silencing and post-transcriptional regulation of gene expression. miRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly(A) tail, and (3) Less efficient translation of the mRNA into proteins by ribosomes. miRNAs are abundant in many mammalian cell types and appear to target about 60% of the genes of humans and other mammals. In the context of the present invention, the provision of human miR-182 (Gene ID: 406958) and miR-183 (Gene ID: 406959) may be particularly advantageous.

The term cell cycle inhibitor relates to molecules of any kind, such as a small chemical molecule, but also proteins, nucleic acids or other molecules, which slow or stop cell cycle progression through various mechanisms. Cell cycle arrest can be induced at different stages, decreasing the rate of cell division and the number of actively cycling cells.

In the context of the present invention, the use of the cell cycle inhibitor AraC is particularly preferred. AraC is also termed cytarabine or cytosine arabinoside and is used as a chemotherapy medication to treat acute myeloid leukemia (AML), acute lymphocytic leukemia (ALL), chronic myelogenous leukemia (CML), and non-Hodgkin's lymphoma. Many cell cycle inhibitors are known in the art and can be identified by a person skilled in the art, including without limitation Pladienolide B, Methotrexate, Roscovitine, Daidzein, Baicalein, Indirubin-3′-oxime, Epothilone B, Narciclasine, AZD 5438, ABT 751, YC 1, 10058-F4, 8-Chloroadenosine, DIM, Plumbagin, Pyridostatin pentahydrochloride, SKPin Cl, CPI 203, CGP 60474, XL 413 hydrochloride, CHMFL-FLT3-122, Potent and selective FLT3 inhibitor, WYE 687 dihydrochloride, NSC 23005 sodium.

Administration of a cell cycle inhibitor relates to addition of the molecule to the cell culture medium, in cases where the molecule becomes available to the cells in this way. The term administration also comprises all kinds of provision of a factor, as described herein in the sense of making the factor available inside the cell to be treated, such as the initial cell of the invention. A provided factor may therefore also be a cell cycle inhibitor.

In embodiments of the method of the invention, the initial cells are cultivated on a basement membrane-like matrix, such as for example Matrigel or another gelatinous protein mixture, such as specific collagen or laminin molecules that support development or maintenance of photoreceptor cells.

Matrigel a gelatinous protein mixture secreted by Engelbreth-Holm-Swarm (EHS) mouse sarcoma cells. Matrigel resembles the complex extracellular environment found in many tissues and is used by cell biologists as a substrate (basement membrane matrix) for culturing cells. Similarly, it is possible to provide different gelatinous protein mixtures for specific cell culture systems that provide a favorable microenvironment for the cultured cells, in the case of the present invention an environment that promotes differentiation of the initial cells towards a photoreceptor-like phenotype. This may be achieved by providing a matrix comprising specific laminins or other extracellular matrix proteins that are abundant in the retina extracellular matrix. In particular, the matrix for culturing the cells of the invention and performing the method of the invention may comprise poly-L-Ornithine, poly-L-Lysine, poly-D-Lysine and/or laminins (In), preferably laminins with a R-2 chain like In323, In423, In523 and/or In521.

In the context of the invention, the term “photoreceptor reporter system” relates to any kind of system that can be used to determine development of a photoreceptor-like phenotype indicating differentiation of the initial cell to a photoreceptor cell or progenitor thereof. Such systems usually employ exogenous nucleic acid sequences encoding for a report gene or a marker gene. Such reporter genes can preferably code fluorescent proteins, which can be easily detected upon expression by standard techniques such as microscopy, cytometry or others. The expression of such reporter or marker genes may be under the control of a genetic element, such as a promoter sequence of a gene that is typically expressed in a photoreceptor cell or a progenitor thereof, or parts of such a sequence. Examples such photoreceptor specific genes, whose genetic control elements may be used in the context of a photoreceptor reporter system, comprise the genes coding for cone-arrestin, rhodopsin, recoverin, NCAM, OTX, CRX, RCVRN, RHO, OPN1SW, OPN1MW and/or OPN1LW. The skilled person can identify further suitable promoter sequences by identifying photoreceptor-specific genes or combination of such genes by looking at typical gene expression profiles of photoreceptor cells that are available in the art. Design of cell type specific reporter system is a well-defined technology known to the skilled person. Marker genes can also encode for proteins that provide resistance to a chemical compound, such as an antibiotic, making it possible to select cells from a mixed culture system that express such a marker under the control of a photoreceptor-specific promoter sequence in the presence of the chemical compound, while the other cells cannot survive in the presence of the respective chemical compound.

Further ways of identifying induced photoreceptor cells in a mixed culture comprising the initial cells may be detection of loss of markers of the initial cell, for example loss of Tra1-60 expression in case of iPSC as initial cells. Cells may be characterized and induced photoreceptor cells may be identified and isolated by means of flow cytometry using expression of fluorescence marker proteins and/or typical surface protein expression patterns of photoreceptor cells and their progenitors in comparison to surface marker patterns of the initial cells.

In the context of the present invention, the term retinopathy relates to any damage to the retina of the eyes, which may cause vision impairment. Retinopathy often refers to retinal vascular disease, or damage to the retina caused by abnormal blood flow. Age-related macular degeneration is included under the umbrella term retinopathy. Retinopathy includes retinal vascular disease and can be broadly categorized into proliferative and non-proliferative types. Frequently, retinopathy is an ocular manifestation of systemic disease as seen in diabetes or hypertension.

Retinopathy further relates to macular degeneration, also known as age-related macular degeneration (AMD or ARMD), which is a medical condition that may result in blurred or no vision in the center of the visual field. Over time, patients may experience a gradual worsening of vision that may affect one or both eyes. While it does not result in complete blindness, loss of central vision can make it hard to recognize faces, drive, read, or perform other activities of daily life. Visual hallucinations may also occur but these do not represent a mental illness. Macular degeneration typically occurs in older people, while genetic factors and smoking also play a role. It appears to be due to damage to the macula of the retina. The severity is divided into early, intermediate, and late types, which may all be treated by use of the cells of the invention. The late type is additionally divided into “dry” and “wet” forms with the dry form making up 90% of cases, wherein all types may be treated by transplantation of cells of the invention.

Retinal degeneration is a retinopathy which consists in the deterioration of the retina caused by the progressive death of its cells. There are several reasons for retinal degeneration, including artery or vein occlusion, diabetic retinopathy, R.L.F./R.O.P. (retrolental fibroplasia/retinopathy of prematurity), or disease (usually hereditary), which may present in many different ways such as impaired vision, night blindness, retinal detachment, light sensitivity, tunnel vision, and loss of peripheral vision to total loss of vision. Of the retinal degenerative diseases retinitis pigmentosa (RP) is a very important example. Inherited retinal degenerative disorders in humans exhibit genetic and phenotypic heterogeneity in their underlying causes and clinical outcomes. A wide variety of causes have been attributed to retinal degeneration, such as disruption of genes that are involved in phototransduction, biosynthesis and folding of the rhodopsin molecule, and the structural support of the retina. Mutations in the rhodopsin gene account for about 25% to 30% of all cases of autosomal dominant retinitis pigmentosa (adRP) in North America. There are many mechanisms of retinal degeneration attributed to rhodopsin mutations or mutations that involve or affect the function of rhodopsin. One mechanism of retinal degeneration is rhodopsin overexpression. Another mechanism, whereby a mutation caused a truncated rhodopsin, was found to affect rod function and increased the rate of photoreceptor degeneration.

Cell transplantation is a novel therapeutic strategy to restore visual responses to the degenerate adult neural retina and it has been shown that transplanted postmitotic photoreceptor precursors are able to functionally integrate into the adult mouse neural retina.

### FIGURES

The invention is further described by the following figures. These are not intended to limit the scope of the invention but represent preferred embodiments or aspects of the invention provided for greater illustration.

### BRIEF DESCRIPTION OF THE FIGURES

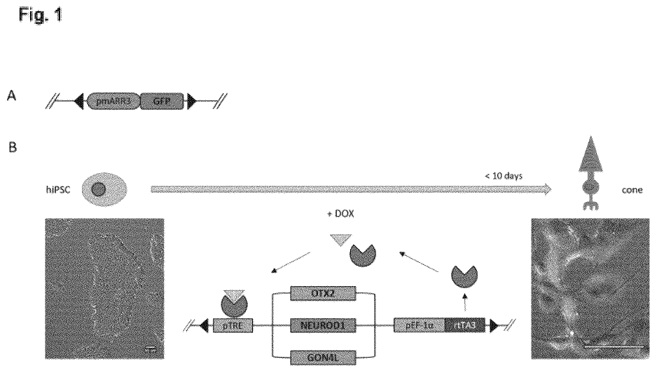

FIG. 1: In vitro photoreceptor differentiation from hiPSCs by over-expressing transcription factors.

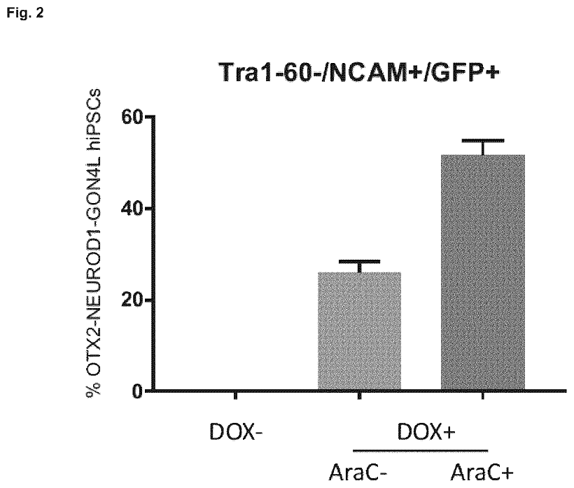

FIG. 2: Flow cytometry analysis of overexpression of a transcription factor combination OTX2, NEUROD1 and GON4L in human iPSC.

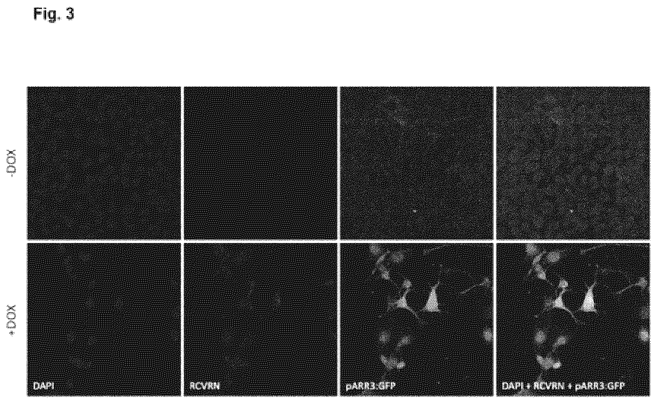

FIG. 3: Microscopy analysis of overexpression of a transcription factor combination OTX2, NEUROD1 and GON4L in human iPSC. S37 and S36.

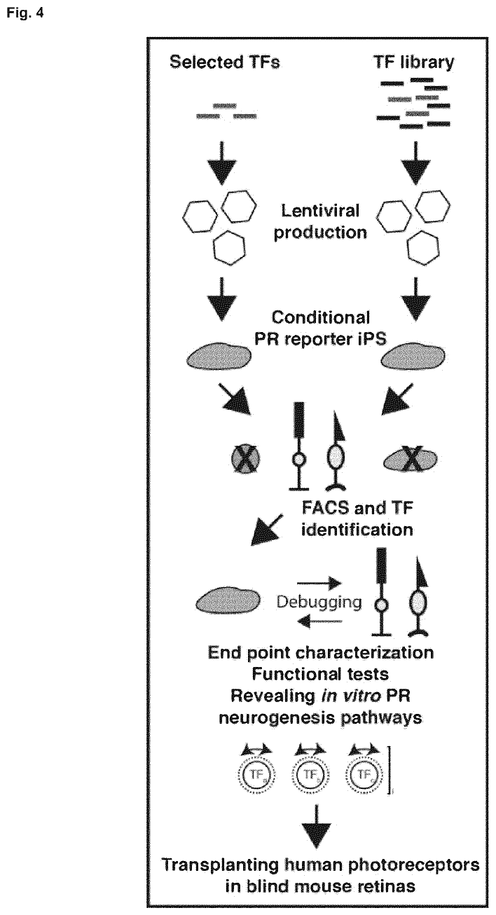

FIG. 4: Scheme of generating induced photoreceptors through TF induction.

FIG. 5: Fluorescence-activated cell sorting plot of induced photoreceptor cells.

FIG. 6: Photoreceptor-specific gene profile of fluorescent cells expressing GON4L.

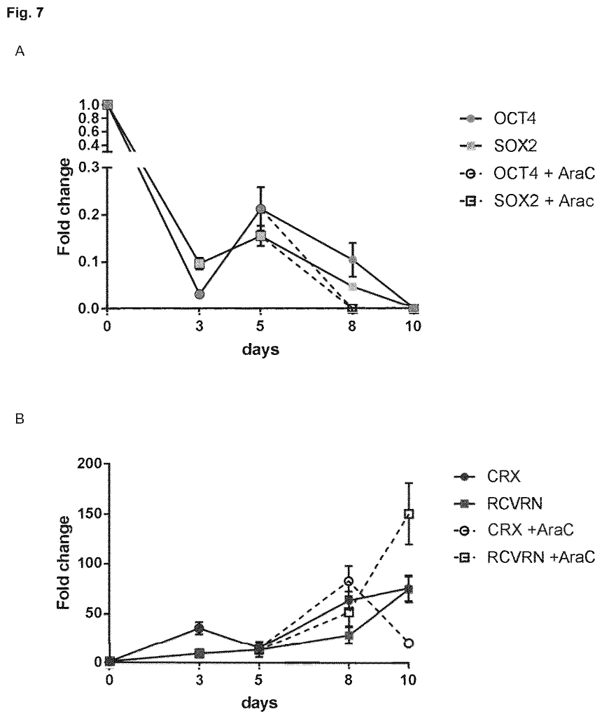

FIG. 7: PGP1cR-ONG cells downregulate stem cell markers (A) and upregulate photoreceptor-specific markers (B) as analyzed by qPCR.

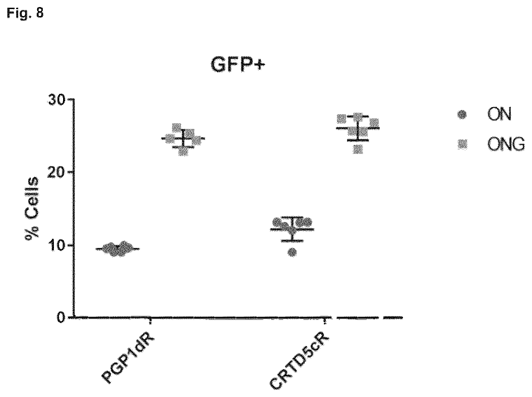

FIG. 8: Flow cytometry analysis of Tra1-60−/NCAM+/GFP+ cells after 5 days of OTX2-NEUROD1 or OTX2-NEUROD1-GON4L overexpression in PGP1 and CRTD5 cone reporter hiPSC lines.

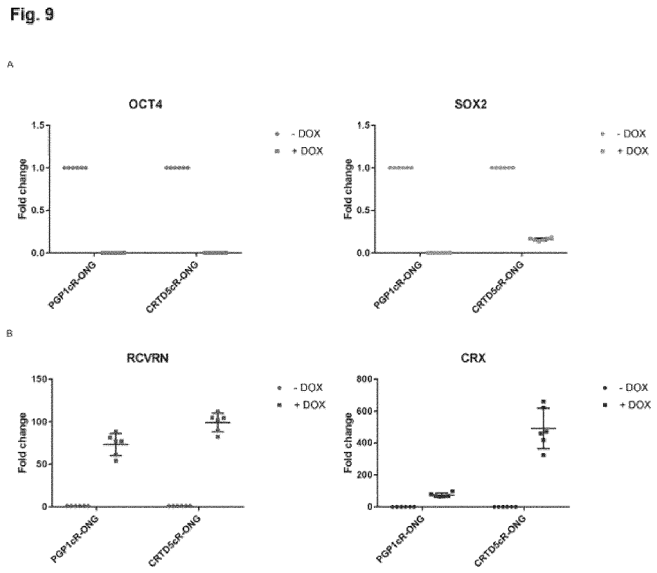

FIG. 9: Overexpression of the TF combination OTX2-NEUROD1-GON4L in hiPSC lines leads to downregulation of pluripotency markers and upregulation of photoreceptor-specific markers as analyzed by qPCR.

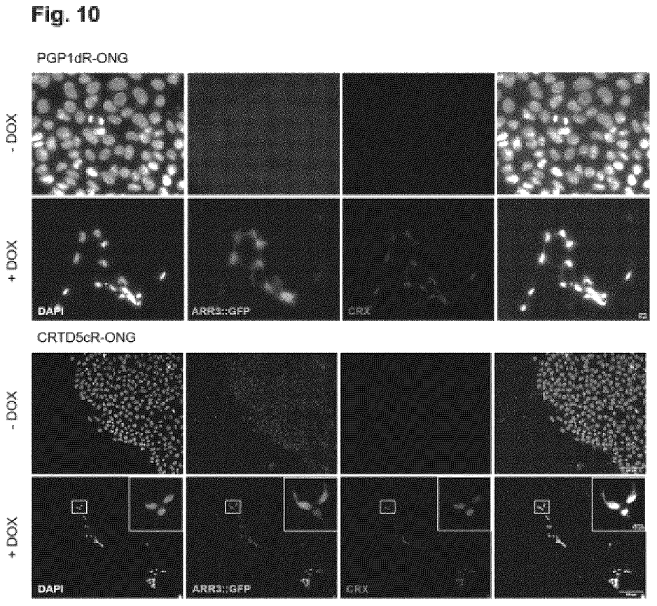

FIG. 10: Overexpression of the TF combination OTX2-NEUROD1-GON4L leads to the upregulation of photoreceptor specific markers as analyzed by immuno-fluorescence.

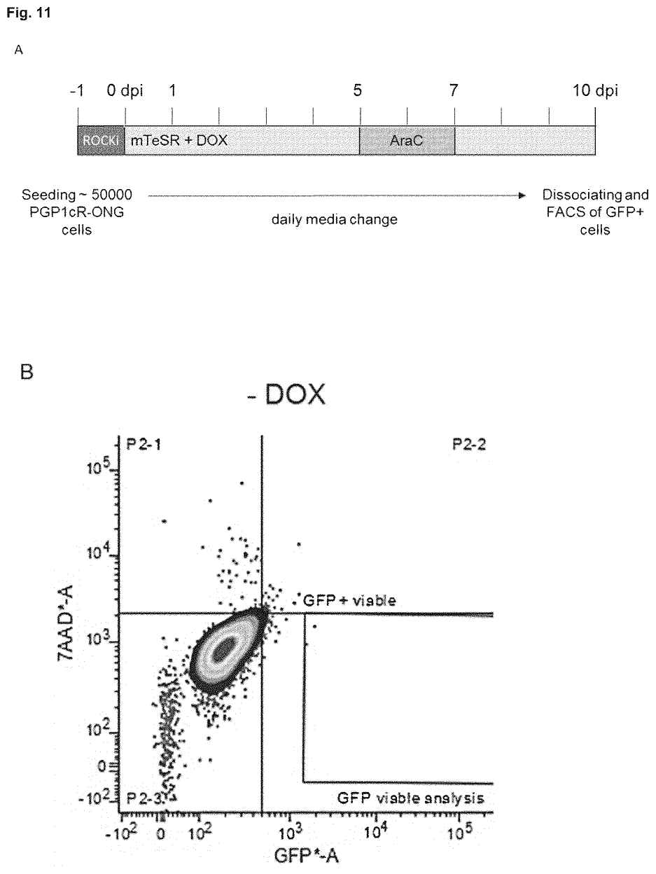

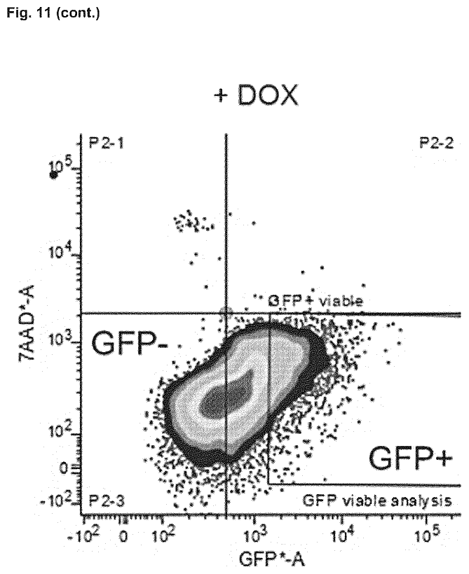

FIG. 11: 10-day differentiation protocol for obtaining cells for transplantation experiments.

### DETAILED DESCRIPTION OF THE FIGURES

FIG. 1: (A) Scheme of the cone reporter cassette introduced into human iPSCs. GFP is under the cone-arrestin promoter, active only in cone photoreceptors. (B) Scheme of the cone differentiation protocol. TFs are under the doxycycline (DOX)-inducible promotor pTRE. When DOX is present, it binds to the transactivator rtTA3 and initiates TFs expression. Less than 10 days of DOX treatment is enough to obtain cone photoreceptors in our 2D cultures. Scale bars, 50 m.

FIG. 2: Overexpression of a transcription factor combination OTX2, NEUROD1 and GON4L for 10 days leads to a differentiation of human induced pluripotent stem cells into 26.1% cone photoreceptor-like cells. By treating them with a cell cycle inhibitor AraC at day 5, we are able to remove a pool of proliferating progenitors and increase the differentiation efficiency to 51.6% (mean, n=3).

FIG. 3: Overexpression of the transcription factor combination OTX2, NEUROD1 and GON4L (ONG) for 7 days in the presence of doxycycline (+DOX) in human induced pluripotent stem cells leads to the upregulation of photoreceptor specific markers. Cells positive for GFP (driven by the cone-arrestin promoter) co-express the photoreceptor precursor marker recoverin (RCVRN, red), indicating their differentiation towards cone photoreceptor-like cells. In our protocol, cells are cultured on Matrigel (protein mixture secreted by mouse sarcoma cells), although photoreceptor-specific laminins might be required to obtain an improved photoreceptor-specific cell morphology. Nonetheless, using the present culture conditions neurite outgrowth, which is a core feature of developing neurons, was observed.

FIG. 4: Selected sets (left) or a library of TFs (right) were induced in human iPS cell lines bearing photoreceptor (PR)-specific fluorescent reporters. In-depth analysis and comparison with in vivo PRs allows a sophisticated assessment of the generated induced PR.

FIG. 5: Fluorescence-activated cell sorting plot of induced photoreceptor cells. Out of 87 fluorescent cells, 85 showed green and 2 cells red fluorescence.

FIG. 6: All cells expressing GON4L were positive for photoreceptor progenitor and precursor markers CRX and OTX2, 8 cells were positive for pan-photoreceptor marker RCVRN, and one FACS sorted cell was positive for late cone marker OPN1SW. Cells were co-expressing different transcription factors from the biased group, among which 6 were co-expressing OTX2 and 3 were found to co-express NEUROD1.

FIG. 7: PGP1cR-ONG cells downregulate stem cell markers (A) and upregulate photoreceptor-specific markers (B) during the 10-day differentiation protocol. (A) Stem cell markers OCT4 and SOX2 are heavily downregulated already after 3 days of OTX2, NEUROD1 and GON4L overexpression indicating that most of the cells are leaving the cell cycle. After 10 days of DOX treatment, or after 8 days if treated with the cell cycle inhibitor AraC, stem cell markers expression levels are mostly undetectable and cells are fully differentiated. (B) Photoreceptor markers CRX and RCVRN are getting upregulated during the course of differentiation and are reaching the expression peak at the day 10. If AraC is added, the remaining cells are upregulating the late photoreceptor marker RCVRN at the expense of the early photoreceptor marker CRX.

FIG. 8: Flow cytometry analysis of Tra1-60−/NCAM+/GFP+ cells after 5 days of OTX2-NEUROD1 (ON) or OTX2-NEUROD1-GON4L (ONG) overexpression in PGP1 and CRTD5 cone reporter hiPSC lines. The TF combination OTX2-NEUROD1 leads to GFP expression from the cone arrestin promoter in ~10% of the differentiated cells. The photoreceptor differentiation efficiency rises to ~25% if GON4L is present. The same differentiation efficiencies can be reproduced in CRTD5cR cells, another cone reporter hiPSC line.

FIG. 9: Overexpression of the TF combination OTX2-NEUROD1-GON4L for 10 days leads to downregulation of pluripotency markers OCT4 and SOX2 (A) and upregulation of photoreceptor-specific markers RCVRN and CRX (B) both in PGP1 and CRTD5 cone reporter hiPSC line. Expression levels of pluripotency markers and the late photoreceptor marker RCVRN are comparable between the two hiPSC lines.

FIG. 10: Overexpression of the TF combination OTX2-NEUROD1-GON4L for 10 days in PGP1 and CRTD5 cone reporter hiPSCs leads to the upregulation of photoreceptor specific markers. Cells positive for GFP (driven by the cone arrestin promoter) co-express the photoreceptor precursor marker CRX (magenta), indicating their differentiation towards cone photoreceptor-like cells. Neurite outgrowth, which is a core feature of developing neurons, was observed.

FIG. 11: 10-day differentiation protocol for obtaining cells for transplantation experiments. (A) Cells are seeded in mTeSR with ROCKi. DOX is added the following day to start the OTX2-NEUROD1-GON4L overexpression and photoreceptor differentiation. The cell cycle inhibitor AraC is added from 5 dpi to 7 dpi to remove any potentially proliferating cells. At 10 dpi, cells are collected using the papain dissociation kit (Worthington Biochemical Corporation) and (B) FACS sorted for live (7-AAD−) GFP+ cells. 150000 cells are then transplanted subretinally following the published protocol (Santos-Ferreira et al. Daylight vision repair by cell transplantation. Stem Cells. 2015 January; 33(1):79-90. doi: 10.1002/stem.1824).

### Examples

The invention is further described by the following examples. These are not intended to limit the scope of the invention but represent preferred embodiments or aspects of the invention provided for greater illustration.

While it is possible to obtain photoreceptors by direct reprogramming from fibroblasts in low quantities, efficient 2D protocols to generate photoreceptors in vitro from human induced pluripotent stem cells (hiPSCs) needs to be established. Forward programming relies on a transcription factors' (TF) abilities to activate distinct differentiation pathways in stem cells. Aiming at finding a TF combination that drives efficient differentiation of stem cells into photoreceptors, we performed a TF-library on library screen.

### Methods

### General Procedure

A TF library consisting of 1748 human TFs was used to generate specific retinal cell types: rod and cone photoreceptors. Photoreceptor-specific reporter constructs were used that become activated at specific states of photoreceptor development (examples: retina and anterior neural fold homeobox (RX), cone-rod homeobox (CRX), cone arrestin-3 (CAR), rhodopsin (RHO)) and induce the expression of fluorescent proteins and a selection marker from a different ubiquitous promoter. In some cases, multiple reporter cassettes were integrated into one iPS cell line via lentiviral gene transfer. Further reporter cell lines were generated by introduction of reporter cassettes using the PiggyBac system. Also, corresponding knock-in cell lines were generated. These reporter human iPS cell lines were tested in retinal organoids for expression, and the best-performing cell line was selected to apply the TF library. Upon TF induction, we screened for fluorescently labeled photoreceptors (CAR and RHO) and/or their precursor cells (RX and CRX) (FIG. 4, right). In parallel, we use the existing knowledge of TFs acting during photoreceptor development, and specifically applied these selected TFs in a biased approach (FIG. 4, left). For the latter experiment, we induced RX, SIX3, SIX6, LHX2, TLL, OTX2, PAX6, SOX1, SOX2, CRX, ONECUT1, VSX2, NRL, TRB2, NEUROD1, NR2E3, RXRG, and RORB (8): these were PCR-amplified from the library pool and were applied individually and also in combinations. We also combined the two library approaches. We generated transcriptomic data from both approaches to minimize the risks of failure and to identify limiting developmental steps. We compare the transcriptomic profiles and genetic programs which result in photoreceptors. Profiles from intermediate but stalled photoreceptor precursor cells are particularly interesting for identifying and debugging critical developmental steps and the pitfalls of stem cell-derived photoreceptor generation.

Rod and cone photoreceptors can be easily distinguished by their specific gene expression profiles. In general, these cell types are well characterized in vivo and, therefore, we can perform comparative troubleshooting. For cellular characterization, we apply specific antibodies against phototransduction cascade members, as well as functional patch-clamp recordings, to characterize the induced photoreceptors. We have previously shown that the upregulation of two microRNAs (miR-182 and miR-183) in photoreceptors of stem cell-derived retinas is sufficient to promote the formation of light-sensitive compartments (outer segments) (11). Hence, the overexpression of these non-coding RNAs is beneficial for functional maturation of photoreceptors.

### Specific Experiment

A reporter hiPSC line was transduced with the lentiviral library of 16 known TFs and subsequently with a comprehensive library consisting of 1748 human TFs. hiPSCs with no TFs were killed by selection using a marker that was included in the lentiviral cassettes. A fraction of the cells was used for TF induction through treatment with doxycycline (dox) for 10 days. Of the transduced and induced cells, 87 were fluorescently labelled and sorted into individual wells (FIG. 5). The RNA of the single cells was extracted, split for single cell qPCR analysis and for the detection of the overexpressed TFs. In particular OTX, CRX, RCVRN, RHO, OPNISW and OPN1 LW, were identified by using specific RT primer for the overexpressed TFs. TF detection was performed by amplifying the TF from cDNA by PCR, loading a gel and excising and sequencing the amplified DNA-bands. Based on the identification of the overexpressed TFs, the preferred TF of the present invention, in particular GON4L, NEUROD1 and OTX2 were identified as being particularly efficient for inducing a cone-phenotype (FIG. 6). TF combinations were validated in the hiPSC reporter line using flow cytometry detecting the loss of a pluripotency marker (Tra1-60) and upregulation of neuronal markers (NCAM) and fluorescence from the reporter cassette.

### Nucleic Acid Sequences Encoding the TFs Used

Nucleic acid sequences encoding the TF GON4L, NEUROD1 and OTX2 as used in the presented experiments are the sequences according to SEQ ID No. 9, SEQ ID No. 11 and SEQ ID No.13, as listed in Table 2. Please note that all three TFs have a V5 tag at their 3′ end.

### Cell Culture

PGP1 (GM23338, Coriell), ATCC DYS0100 (ATCC® ACS-1019™, ATCC) and CRTD5 (reprogrammed at CRTD iPSC facility, Kutsche et al. Cell Systems 2018, Oct. 24; 7(4):438-452) human induced pluripotent stem cells (hiPSCs) were cultured in mTeSR1 media (05850, StemCell Technologies). Before adding hiPSCs, regular tissue culture well plates were coated with hESC-qualified Matrigel matrix (354277, Corning) and incubated for 60 min at room temperature. The hiPSCs were cultured under standard conditions (5% CO2, 37° C.) and mTeSR1 media was exchanged daily. For passaging, hiPSCs were dissociated from the wells by adding TrypLE Express (12604013, Thermo Fisher Scientific), washed with phosphate-buffered saline (PBS, pH 7.2; 14190169, Thermo Fisher Scientific), spun down at 400×g and added to fresh Matrigel-coated tissue culture wells in mTeSR1 media with 3 g/ml InSolution Y-27632 rho kinase inhibitor (688001, Merck Millipore). Alternatively, cells were frozen in mFreSR media (05854, StemCell Technologies).

Stable integration of an inducible TF or photoreceptor reporter cassette was done by using the PiggyBac transposon system. All vector elements between the 5′ core insulator and the SV40 polyA site of the PiggyBac vector backbone PB-TRE-dCas9-VPR13 (Addgene plasmid #63800; Chavez et al., 2015, Nat Methods. 2015 Mar. 2. doi: 10.1038/nmeth.3312) were replaced with corresponding DNA fragments. 10 g of the plasmid were mixed with 2 g of Super PiggyBac Transposase Expression Vector (PB210PA-1-S, Biocat) and electroporated to hiPSCs with the Lonza 4D X-unit, pulse CB-156 and the P3 Primary Cell 4D-Nucleofector Kit L (V4XP-3024, Lonza). According to the chosen selection cassette, Blasticidin (25 g/ml), Puromycin (0.5-1 μg/ml) or Hygromycin B (250 μl/ml) were applied.

Standard lentiviral transduction was performed for the TF screen. Cell numbers and viral particles were adjusted to obtain a multiplicity of infection of 1. PGP1 iPSCs containing the photoreceptor reporter cassette were serially transduced with either the unbiased TF library (1748 TFs each included in the lentiviral pLIX_403 backbone (Addgene plasmid 41395)) or the library of selected TFs (backbone from Addgene plasmid 61473) and subsequently selected by corresponding selection markers.

TF Induction to Differentiate hiPSC:

Transcription from the TeTOn promoter was induced by the application of 0.5 g/ml doxycycline (D9891, Sigma-Aldrich) into mTeSR1 media.

### Details of the Photoreceptor Reporter System

The photoreceptor reporter system is based on the PiggyBac vector PB-TRE-dCas9-VPR13 (Addgene plasmid #63800; Chavez et al., 2015, Nat Methods. 2015 Mar. 2. doi: 10.1038/nmeth.3312). All vector elements between the 5′ core insulator and the SV40 polyA site were replaced by an eGFP cassette driven from the mouse cone arrestin promoter (mCAR, Busskamp et al. Science 2010, Jul. 23; 329(5990):413-7) or by a human Rhodopsin promoter (RHO, Busskamp et al. Science 2010, Jul. 23; 329(5990):413-7) driving the red-fluorescent protein dsRED. Downstream of the fluorescent proteins, a Woodchuck hepatitis virus posttranscriptional regulatory element (WPRE) and a blasticin selection cassette driven from the ubiquitin C promoter (both taken from Addgene plasmid 61473) were added. The corresponding vectors pb-mCAR-EGFP-UBC-Blasti and pb-Rho-dsRed-UBC-Blasti were co-nucleofected into PGP1 hiPSCs and selected for transgenic clones with both constructs integrated.

### Concentrations of the Cell Cycle Inhibitor and Other Reagents Used in the Respective Experiments.

Cytosine β-D-arabinofuranoside hydrochloride (Ara-C, C6645, Sigma) was used at a final concentration of 5 M for 24 h to deplete dividing cells in neuronal cultures.

### Results

87% of the sorted cells were qPCR-positive for at least one of the tested photoreceptor-specific genes indicating the cell-type-precision of our screen. Some of the tested TF combinations comprising GON4L and in some cases also OTX2 and NEUROD1 led to a significant loss of the pluripotency marker Tra1-60 and upregulation of a neuronal marker NCAM (hiPSCs: 0.47±0.07%, hiPSCs-TFs: 75.23±3.7%; mean±SEM, Welch's two-tailed t-test; p=0.002) after 5 days of overexpression, indicating that cells are differentiating towards the neuronal lineage. Furthermore, fluorescence microscopy and flow cytometry detected GFP-positive cells after 10 days suggesting the presence of cone photoreceptors.

### CONCLUSION

We systematically screened TFs based from in vivo studies and a human TF library to find the combination that would help us reaching a final goal of engineering human photoreceptors in vitro. Our data suggest that the known factors were insufficient to drive photoreceptor differentiation, indicating that photoreceptor genesis from hiPSCs requires additional TFs, in particular GON4L. The combination of GON4L with OTX2 and NEUROD1 was particularly advantageous for efficient induction of photoreceptor differentiation. In-vitro-engineered photoreceptors might serve as a donor material for cell transplantation to treat blindness as sufficient quantities can be generated within 10 days compared to hundreds of days if dissociated from 3D human retinal organoids.

Transplanting Induced Human Photoreceptors into Blind Mouse Retinas

As previously mentioned, there are many approaches in which photoreceptors are transplanted into mouse models of retinal degeneration. For this purpose, mouse photoreceptor progenitors can be taken and injected into the subretinal space (12-14) of blind retinas. In addition, rod photoreceptor precursor cells derived from 3d organoids can be isolated and successfully transplanted (15). A low fraction of these mouse cells has been shown to functionally integrate into the retina of host mice. Induced human photoreceptors haven't been used before, and we will therefore be the first to use these cells as starting material for transplantation into the retinas of blind mice.

To visualize and functionally test transplanted photoreceptors, we plan to tag these cells prior to injection with fluorescent reporters that are fused to hyperpolarizing optogenetic tools (16, 17). In addition to fluorescent detection, we will trigger light sensitivity by stimulating the optogenetic tool with light, and subsequently record the light responses. Since intrinsic photoreceptors in disease mouse models are insensitive to light, all light responses can be tracked back to transplanted, and therefore functionally integrated, cells. The intrinsic phototransduction cascades of rods and cones are log-units more sensitive than the optogenetic sensors. Hence, by controlling the light levels for stimulation, discrimination between intrinsic and optogenetic photoresponses will be possible.

To measure the success of reactivation, we will perform patch-clamp recordings directly from the transplanted photoreceptors. To test whether the cones integrate into existing retinal circuits, we will record by patch clamp or MEA from retinal ganglion cells. Recovered vision will also be investigated using behavioral tests as shown before (16). In addition to the functional studies, we will perform immunohistochemical analyses followed by confocal and electron microscopy at the CRTD light microscopy facility. We will also study the transcriptomic profiles of successfully integrated human photoreceptors and compare them to the ones that failed, in order to determine the limiting biological parameters to improve cone integration.

### REFERENCES

1. Ishii, T., Yin, C., Seko, Y., Umezawa, A. & Kaneda, M. Variation in the Phenotype of Photosensitive Cells Produced from Human Fibroblast Cell Lines. 1 Nippon Med Sch 85, 110-116 (2018).
2. Seko, Y. et al. Derivation of human differential photoreceptor cells from adult human dermal fibroblasts by defined combinations of CRX, RAX, OTX2 and NEUROD. Genes Cells 19, 198-208 (2014).
3. Gonzalez-Cordero, A. et al. Recapitulation of Human Retinal Development from Human Pluripotent Stern Cells Generates Transplantable Populations of Cone Photoreceptors. Stern Cell Reports 9, 820-837 (2017).
4. Slembrouck-Brec, A., Nanteau, C., Sahel, J. A., Goureau, 0. & Reichman, S. Defined Xeno-free and Feeder-free Culture Conditions for the Generation of Human IPSC-derived Retina! Cell Models. 1 Vis Exp (2018).
5. Volkner, M., Kurth, T. & Karl, M. O. The Mouse Retinal Organoid Trisection Recipe: Efficient Generation of 3D Retinal Tissue from Mouse Embryonic Stern Cells. Methods Mol Bio11834, 119-141 (2019).
6. Volkner, M. et al. Retinal Organoids from Pluripotent Stern Cells Efficiently Recapitulate Retinogenesis. Stern Cell Reports 6, 525-538 (2016).
7. Lakowski, 1. et al. Isolation of Human Photoreceptor Precursors via a Cell Surface Marker Panel from Stern Cell-Derived Retina! Organoids and Fetal Retinae. Stern Cells 36, 709-722 (2018).
8. Hennig, A. K., G. H. Peng, and S. Chen, Brain Res, 2008 Feb. 4; 1192:114-33.
9. Zuber, M. E., Curr Top Dev Biol, 2010; 93:29-60.
10. Zuber, M. E., et al., Development, 2003, November; 130(21):5155-67.
11. Busskamp, V., et al., Neuron, 2014, Aug. 6; 83(3):586-600.
12. MacLaren, R. E., et al., Nature, 2006, Nov. 9; 444(7116):203-7.
13. Pearson, R. A., et al., Nature, 2012, May 3; 485(7396):99-103.
14. Santos-Ferreira, T., et al., Stem Cells, 2015 January; 33(1):79-90.
15. Gonzalez-Cordero, A., et al., Nat Biotechnol, 2013 August; 31(8):741-7.
16. Busskamp, V., et al., Science, 2010, Jul. 23; 329(5990):413-7.
17. Chuong, A. S., et al., Nat Neurosci, 2014, August; 17(8):1123-9.

The foregoing description is illustrative of particular embodiments of the invention but is not meant to be a limitation upon the practice thereof. The following claims, including all equivalents thereof are intended to define the scope of the invention.

1. A method for producing an induced photoreceptor cell from an initial cell, the method comprising:
providing transcription factors (TFs) comprising GON4L, OTX2, and NEUROD1 to the initial cell, wherein the initial cell is one of a pluripotent or multipotent mammalian cell, a fibroblast, a retinal progenitor cell (RPC), a retinal pigment epithelium (RPE) cell, a Mueller Glia cell or other cell found in the eye or retina with the proviso that the initial cell is not a photoreceptor cell; and
allowing sufficient time to induce a reprogramming of the initial cell to the induced photoreceptor cell.

2. The method according to claim 1, wherein the TFs are expressed from one or more exogenous nucleic acid molecules within the initial cell.

3. The method according to claim 1, comprising administering a cell cycle inhibitor to the initial cell.

4. The method according to claim 1, wherein an induced photoreceptor cell produced from the initial cell is determined by a photoreceptor reporter system present in the initial cell.

5. The method according to claim 1, wherein the TFs are expressed to a level greater than in an iPSC.

6. The method according to claim 1, wherein the TFs are expressed from one or more viral vectors.

7. The method according to claim 6, wherein the cell cycle inhibitor is AraC.

8. The method according to claim 7, wherein the photoreceptor reporter system comprises one or more photoreceptor-specific promoter sequences and one or more reporter genes and/or selection markers.

9. Induced photoreceptor cell obtainable by the method according to claim 1, wherein said cells comprise one or more exogenous nucleic acid molecules encoding the transcription factors (TFs) GON4L, OTX2 and NEUROD1.

10. A kit for producing induced photoreceptor cells from an initial cell according to the method of claim 1, comprising
a vector system for providing GON4L, OTX2, and NEUROD1 and optionally further TFs to the initial cell,
reagents for detecting induced photoreceptor cells generated from an initial cell, such as
a photoreceptor-specific reporter system,
antibodies for detection of photoreceptor marker proteins, and/or
primers for detection of OTX, CRX, RCVRN, RHO, OPN1SW, OPN1MW and/or OPN1LW mRNA by PCR, and
optionally a cell cycle inhibitor.

11. An expression vector system comprising one or more nucleic acid sequences operably coupled to one or more promoters, wherein said sequences encode one or more transcription factors (TFs) comprising at least GON4L, OTX2, NEUROD1, and optionally miR-182 and/or miR-183.


SEQUENCE LISTING (13 sequences)

SEQ ID NO: 1 - protein, 1098 aa, Homo sapiens [sequence omitted]

SEQ ID NO: 2 - protein, 2241 aa, Homo sapiens
MLPCKKRRTTVTESLQHKGNQEENNVDLESAVKPESDQVKDLSSVSLSWDPSHGRVAGFEVQSLQDAGNQLGMEDTSLSSGMLTQNTNVPILEGVDVAISQGITLPSLESFHPLNIHIGKGKLHATGSKRGKKMTLRPGPVTQEDRCDHLTLKEPFSGEPSEEVKEEGGKPQMNSEGEIPSLPSGSQSAKPVSQPRKSTQPDVCASPQEKPLRTLFHQPEEEIEDGGLFIPMEEQDNEESEKRRKKKKGTKRKRDGRGQEGTLAYDLKLDDMLDRTLEDGAKQHNLTAVNVRNILHEVITNEHVVAMMKAAISETEDMPMFEPKMTRSKLKEVVEKGVVIPTWNISPIKKANEIKPPQFVDIHLEEDDSSDEEYQPDDEEEDETAEESLLESDVESTASSPRGAKKSRLRQSSEMTETDEESGILSEAEKVTTPAIRHISAEVVPMGPPPPPKPKQTRDSTFMEKLHAVDEELASSPVCMDSFQPMDDSLIAFRTRSKMPLKDVPLGQLEAELQAPDITPDMYDPNTADDEDWKMWLGGLMNDDVGNEDEADDDDDPEYNFLEDLDEPDTEDFRTDRAVRITKKEVNELMEELFETFQDEMGFSNMEDDGPEEEECVAEPRPNFNTPQALRFEEPLANLLNEQHRTVKELFEQLKMKKSSAKQLQEVEKVKPQSEKVHQTLILDPAQRKRLQQQMQQHVQLLTQIHLLATCNPNLNPEATTTRIFLKELGTFAQSSIALHHQYNPKFQTLFQPCNLMGAMQLIEDFSTHVSIDCSPHKTVKKTANEFPCLPKQVAWILATSKVFMYPELLPVCSLKAKNPQDKIVFTKAEDNLLALGLKHFEGTEFPNPLISKYLLTCKTAHQLTVRIKNLNMNRAPDNIIKFYKKTKQLPVLGKCCEEIQPHQWKPPIEREEHRLPFWLKASLPSIQEELRHMADGAREVGNMTGTTEINSDRSLEKDNLELGSESRYPLLLPKGVVLKLKPVATRFPRKAWRQKRSSVLKPLLIQPSPSLQPSFNPGKTPARSTHSEAPPSKMVLRIPHPIQPATVLQTVPGVPPLGVSGGESFESPAALPAVPPEARTSFPLSESQTLLSSAPVPKVMLPSLAPSKFRKPYVRRRPSKRRGVKASPCMKPAPVIHHPASVIFTVPATTVKIVSLGGGCNMIQPVNAAVAQSPQTIPITTLLVNPTSFPCPLNQSLVASSVSPLIVSGNSVNLPIPSTPEDKAHVNVDIACAVADGENAFQGLEPKLEPQELSPLSATVFPKVEHSPGPPLADAECQEGLSENSACRWTVVKTEEGRQALEPLPQGIQESLNNPTPGDLEEIVKMEPEEAREEISGSPERDICDDIKVEHAVELDTGAPSEELSSAGEVTKQTVLQKEEERSQPTKTPSSSQEPPDEGTSGTDVNKGSSKNALSSMDPEVRLSSPPGKPEDSSSVDGQSVGTPVGPETGGEKNGPEEEEEEDFDDLTQDEEDEMSSASEESVLSVPELQETMEKLTWLASERRMSQEGESEEENSQEENSEPEEEEEEEAEGMESLQKEDEMTDEAVGDSAEKPPTFASPETAPEVETSRTPPGESIKAAGKGRNNHRARNKRGSRARASKDTSKLLLLYDEDILERDPLREQKDLAFAQAYLTRVREALQHIPGKYEDFLQVIYEFESSTQRRTAVDLYKSLQILLQDWPQLLKDFAAFLLPEQALACGLFEEQQAFEKSRKFLRQLEICFAENPSHHQKIIKVLQGCADCLPQEITELKTQMWQLLKGHDHLQDEFSIFFDHLRPAASRMGDFEEINWTEEKEYEFDGFEEVALPDVEEEEEPPKIPTASKNKRKKEIGVQNHDKETEWPDGAKDCACSCHEGGPDSKLKKSKRRSCSHCSSKVCDSKSYKSKEPHELVGSSPHREASPMPGAKEAGQGKDMMEEEAPEERESTEATQSRTVRTTRKGEMPVSAGLAVGSTLPSPREVTVTERLLLDGPPPHSPETPQFPPTTGAVLYTVKRNQVGPEVRSCPKASPRLQKEREGQKAVSESEALMLVWDASETEKLPGTVEPPASFLSPVSSKTRDAGRRHVSGKPDTQERWLPSSRARVKTRDRTCPVHESPSGIDTSETSPKAPRGGLAKDSGTQAKGPEGEQQPKAAEATVCANNSKVSSTGEKVVLWTREADRVILTMCQEQGAQPQTFNIISQQLGNKTPAEVSHRFRELMQLFHTACEASSEDEDDATSTSNADQLSDHGDLLSEEELDE

SEQ ID NO: 3 - protein, 1529 aa, Homo sapiens
  REGION 1..1529: note = misc_feature - human GON4L isoform B, GenBank: AAR01262.1
MLPCKKRRTTVTESLQHKGNQEENNVDLESAVKPESDQVKDLSSVSLSWDPSHGRVAGFEVQSLQDAGNQLGMEDTSLSSGMLTQNTNVPILEGVDVAISQGITLPSLESFHPLNIHIGKGKLHATGSKRGKKMTLRPGPVTQEDRCDHLTLKEPFSGEPSEEVKEEGGKPQMNSEGEIPSLPSGSQSAKPVSQPRKSTQPDVCASPQEKPLRTLFHQPEEEIEDGGLFIPMEEQDNEESEKRRKKKKGTKRKRDGRGQEGTLAYDLKLDDMLDRTLEDGAKQHNLTAVNVRNILHEVITNEHVVAMMKAAISETEDMPMFEPKMTRSKLKEVVEKGVVIPTWNISPIKKANEIKPPQFVDIHLEEDDSSDEEYQPDDEEEDETAEESLLESDVESTASSPRGAKKSRLRQSSEMTETDEESGILSEAEKVTAPAIRHISAEVVPMGPPPPPKPKQTRDSTFMEKLHAVDEELASSPVCMDSFQPMDDSLIAFRTRSKMPLKDVPLGQLEAELQAPDITPDMYDPNTADDEDWKMWLGGLMNDDVGNEDEADDDDDPEYNFLEDLDEPDTEDFRTDRAVRITKKEVNELMEELFETFQDEMGFSNMEDDGPEEEECVAEPRPNFNTPQALRFEEPLANLLNEQHRTVKELFEQLKMKKSSAKQLQEVEKVKPQSEKVHQTLILDPAQRKRLQQQMQQHVQLLTQIHLLATCNPNLNPEATTTRIFLKELGTFAQSSIALHHQYNPKFQTLFQPCNLMGAMQLIEDFSTHVSIDCSPHKTVKKTANEFPCLPKQVAWILATSKVFMYPELLPVCSLKAKNPQDKIVFTKAEDNLLALGLKHFEGTEFPNPLISKYLLTCKTAHQLTVRIKNLNMNRAPDNIIKFYKKTKQLPVLGKCCEEIQPHQWKPPIEREEHRLPFWLKASLPSIQEELRHMADGAREVGNMTGTTEINSDRSLEKDNLELGSESRYPLLLPKGVVLKLKPVATRSPRKAWRQKRSSVLKPLLIQPSPSLQPSFNPGKTPARSTHSEAPPSKMVLRIPHPIQPATVLQTVPGVPPLGVSGGESFESPAALPAVPPEARTSFPLSESQTLLSSAPVPKVMLPSLAPSKFRKPYVRRRPSKRRGVKASPCMKPAPVIHHPASVIFTVPATTVKIVSLGGGCNMIQPVNAAVAQSPQTIPITTLLVNPTSFPCPLNQSLVASSVSPLIVSGNSVNLPIPSTPEDKAHVNVDIACAVADGENAFQGLEPKLEPQELSPLSATVFPKVEHSPGPPLADAECQEGLSENSACRWTVVKTEEGRQALEPLPQGIQESLNNPTPGDLEEIVKMEPEEAREEISGSPERDICDDIKVEHAVELDTGAPSEELSSAGEVTKQTVLQKEEGRSQPTKTPSSSQEPPDEGTSGTDVNKGSSKNALSSMDPEVRLSSPPGKPEDSSSVDGQSVGTPVGPETGGEKNGPEEEEEEDFDDLTQDEEDEMSSASEESVLSVPELQVRAGEYSQVFRGLSNMYHLLICHLLACCTMDSPKIICI

SEQ ID NO: 4 - protein, 2241 aa, Homo sapiens; identical to SEQ ID NO: 2
  REGION 1..2241: note = misc_feature - human GON4L isoform C, GenBank: AAR01261.1

SEQ ID NO: 5 - protein, 356 aa, Homo sapiens
  REGION 1..356: note = misc_feature - human NEUROD1, GenBank: BAJ84018.1
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 6 - protein, 297 aa, Homo sapiens
  REGION 1..297: note = misc_feature - human OTX2 Isoform A, NCBI Reference Sequence: NP_068374.1
MMSYLKQPPYAVNGLSLTTSGMDLLHPSVGYPGPWASCPAATPRKQRRERTTFTRAQLDVLEALFAKTRYPDIFMREEVALKINLPESRVQVWFKNRRAKCRQQQQQQQNGGQNKVRPAKKKTSPAREVSSESGTSGQFTPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 7 - protein, 289 aa, Homo sapiens
  REGION 1..289: note = misc_feature - human OTX2 Isoform B, NCBI Reference Sequence: NP_001257453.1
MMSYLKQPPYAVNGLSLTTSGMDLLHPSVGYPATPRKQRRERTTFTRAQLDVLEALFAKTRYPDIFMREEVALKINLPESRVQVWFKNRRAKCRQQQQQQQNGGQNKVRPAKKKTSPAREVSSESGTSGQFTPPSSTSVPTIASSSAPVSIWSPASISPLSDPLSTSSSCMQRSYPMTYTQASGYSQGYAGSTSYFGGMDCGSYLTPMHHQLPGPGATLSPMGTNAVTSHLNQSPASLSTQGYGASSLGFNSTTDCLDYKDQTASWKLNFNADCLDYKDQTSSWKFQVL

SEQ ID NO: 8 - DNA, 4587 bp, Homo sapiens
  source 1..4587: mol_type = genomic DNA
atgttgccctgtaagaagagaagaactacagtgacagagtccctacagcataaaggcaatcaagaggaaaacaacgtagacctagaatcagccgttaaaccagaatctgaccaggttaaggacttgagttcggtgtcactatcctgggatccaagtcatggcagagtagctggcttcgaagtacagtctttgcaggatgcaggaaatcagcttggtatggaggatacatctctgagctctggaatgctcacccagaacacaaatgtaccaattctagaaggtgttgatgtggccatctctcagggaatcaccctaccttccttggagtcttttcacccccttaatatacacattggtaaaggaaaactccacgctactggctcaaagagagggaaaaaaatgacactcaggcctgggccagttacccaagaagacagatgtgatcatcttaccctaaaggagcctttttcaggagagcctagtgaagaagtcaaggaagaaggagggaaacctcaaatgaattctgaaggggagataccttccctgccatcaggcagccaatctgcaaaaccagtaagccagcccaggaaatcaacccagccagatgtttgtgcctctcctcaagaaaagccactcaggactctgtttcaccaacctgaggaagagatagaagatggtggactcttcattccaatggaagaacaagacaatgaagaaagtgagaaaaggagaaaaaagaaaaagggtaccaagaggaaacgagatggaaggggtcaagaagggaccttggcatatgacctgaaactggatgacatgcttgaccgtaccttggaggatggtgccaagcagcacaatctaacagcagtcaatgtccgaaacatccttcatgaagtaatcacaaatgaacacgtggtagctatgatgaaagcagccatcagtgagacggaagatatgccaatgtttgagcctaaaatgacacgctctaaactgaaggaagtagtggaaaaaggagtggtaattccaacatggaatatttcaccaattaagaaggccaatgaaattaagcctcctcagtttgtggatatccaccttgaagaagatgattcctcagatgaagaataccagccggatgatgaagaagaagatgaaactgctgaagagagcttattggaaagtgatgttgaaagcactgcttcatctccacgtggggcaaagaaatccagattgaggcagtcttctgagatgactgaaacagatgaggagagtggcatattatcagaggctgagaaagtcaccacaccagccatcaggcacatcagtgctgaggtagtgcccatggggcccccgccccctccaaagccgaaacagaccagagatagtactttcatggagaagttacatgcggtagatgaggagctggcttccagtccagtctgcatggattctttccagcccatggatgacagtctcattgcatttcgaacgcgttctaagatgcccctgaaagatgttcccctgggccaattagaggcagagctccaagctccagacatcactccagatatgtatgaccccaatacggcagatgatgaggactggaagatgtggctggggggacttatgaatgatgatgtggggaatgaagatgaagcagatgatgatgatgatccagaatataatttcctggaagacctcgatgaaccagacacagaggatttccggactgaccgggcagtgagaatcaccaaaaaggaagtaaatgagctgatggaagagctgtttgaaactttccaagatgagatgggattctccaacatggaagatgatggcccagaagaggaggagtgtgtagctgagcctcgtcctaactttaacacccctcaagctctacggtttgaggaaccactggccaacctgttaaatgaacaacatcggacagtgaaggagctatttgaacagctgaagatgaagaaatcttcagccaaacagctgcaggaagtagagaaggttaaaccccagagtgagaaagttcatcagactctgattctggacccagcacagaggaagagactccagcagcagatgcagcagcacgttcagctcttgacccaaatccaccttcttgccacctgcaaccccaacctcaatccggaggccactaccaccaggatatttcttaaagagctgggaacctttgctcaaagctccatcgcccttcaccatcagtacaaccccaagtttcagaccctgttccaaccctgtaacttgatgggagctatgcagctgattgaagacttcagcacacatgtcagcattgactgcagccctcataaaactgtcaagaagactgcgaatgaatttccctgtttgccaaagcaagtggcttggattctggccacaagcaaggttttcatgtatccagagttacttccagtgtgttccctgaaggcaaagaatccccaggataagatcgtcttcaccaaggctgaggacaatttgttagctttaggactgaagcattttgaaggaactgagtttcctaatcctctaatcagcaagtaccttctaacctgcaaaactgcccaccaactgacagtgagaatcaagaacctcaacatgaacagagctcctgacaacatcattaaattttataagaagaccaaacagctgccagtcctaggaaaatgctgtgaagagatccagccacatcagtggaagccacctatagagagagaagaacaccggctcccattctggttaaaggccagtctgccatccatccaggaagaactgcggcacatggctgatggtgctagagaggtaggaaatatgactggaaccactgagatcaactcagatcgaagcctagaaaaagacaatttggagttggggagtgaatctcggtacccactgctattgcctaagggtgtagtcctgaaactgaagccagttgccacccgtttccccaggaaggcttggagacagaagcgttcatcagtcctgaagcccctccttatccaacccagcccctctctccagcccagcttcaaccctgggaaaacaccagcccgatcaactcattcagaagcccctccgagcaaaatggtgctccggattcctcacccaatacagccagccactgttttacagacagttccaggtgtccctccactgggggtcagtggaggtgagagttttgagtctcctgcagcactgcctgctgtgccccctgaggccaggacaagcttccctctgtctgagtcccagactttgctctcttctgcccctgtgcccaaggtaatgctgccctcccttgccccttctaagtttcgaaagccatatgtgagacggagaccctcaaagagaagaggagtcaaggcctctccctgtatgaaacctgcccctgttatccaccaccctgcatctgttatcttcactgttcctgctaccactgtgaagattgtgagccttggcggtggctgtaacatgatccagcctgtcaatgcggctgtggcccagagtccccagactattcccatcactaccctcttggttaaccctacttccttcccctgtccattgaaccagtcccttgtggcctcctctgtctcacccttaattgtttctggcaattctgtgaatcttcctataccatccacccctgaagataaggcccacgtgaatgtggacattgcttgtgctgtggctgatggggaaaatgcctttcagggcctagaacccaaattagagccccaggaactatctcctctctctgctactgttttcccgaaagtggaacatagcccagggcctccactagcagatgcagagtgccaagaaggattgtcagagaatagtgcctgtcgctggaccgttgtgaaaacagaggaggggaggcaagctctggagccgctccctcagggcatccaggagtctctaaacaaccctacccctggggatttagaggaaattgtcaagatggaacctgaagaagctagagaggaaatcagtggatcccctgagcgtgatatttgtgatgacatcaaagtggaacatgctgtggaattggacactggtgccccaagcgaggagttgagcagtgctggagaagtaacgaaacagacagtcttacagaaggaagaggagaggagtcagccaactaaaaccccttcatcttctcaagagccccctgatgaaggaacctcagggacagatgtgaacaaaggatcatcaaagaatgctttgtcctcaatggatcctgaagtgaggcttagtagccccccagggaagccagaagattcatccagtgttgatggtcagtcagtggggactccagttgggccagaaactggaggagagaagaatgggccagaagaagaggaagaagaggactttgatgacctcacccaagatgaggaagatgaaatgtcatcagcttctgaggaatctgtgctttctgtcccagaactccaggtgagagctggagaatattctcaagtatttcgtggactcagtaatatgtatcacttattgatatgccacctgcttgcttgctgcactatggatagtcctaaaatcatttgtatt

SEQ ID NO: 9 - DNA, 4641 bp, Homo sapiens
  source 1..4641: mol_type = genomic DNA
atgttgccctgtaagaagagaagaactacagtgacagagtccctacagcataaaggcaatcaagaggaaaacaacgtagacctagaatcagccgttaaaccagaatctgaccaggttaaggacttgagttcggtgtcactatcctgggatccaagtcatggcagagtagctggcttcgaagtacagtctttgcaggatgcaggaaatcagcttggtatggaggatacatctctgagctctggaatgctcacccagaacacaaatgtaccaattctagaaggtgttgatgtggccatctctcagggaatcaccctaccttccttggagtcttttcacccccttaatatacacattggtaaaggaaaactccacgctactggctcaaagagagggaaaaaaatgacactcaggcctgggccagttacccaagaagacagatgtgatcatcttaccctaaaggagcctttttcaggagagcctagtgaagaagtcaaggaagaaggagggaaacctcaaatgaattctgaaggggagataccttccctgccatcaggcagccaatctgcaaaaccagtaagccagcccaggaaatcaacccagccagatgtttgtgcctctcctcaagaaaagccactcaggactctgtttcaccaacctgaggaagagatagaagatggtggactcttcattccaatggaagaacaagacaatgaagaaagtgagaaaaggagaaaaaagaaaaagggtaccaagaggaaacgagatggaaggggtcaagaagggaccttggcatatgacctgaaactggatgacatgcttgaccgtaccttggaggatggtgccaagcagcacaatctaacagcagtcaatgtccgaaacatccttcatgaagtaatcacaaatgaacacgtggtagctatgatgaaagcagccatcagtgagacggaagatatgccaatgtttgagcctaaaatgacacgctctaaactgaaggaagtagtggaaaaaggagtggtaattccaacatggaatatttcaccaattaagaaggccaatgaaattaagcctcctcagtttgtggatatccaccttgaagaagatgattcctcagatgaagaataccagccggatgatgaagaagaagatgaaactgctgaagagagcttattggaaagtgatgttgaaagcactgcttcatctccacgtggggcaaagaaatccagattgaggcagtcttctgagatgactgaaacagatgaggagagtggcatattatcagaggctgagaaagtcaccacaccagccatcaggcacatcagtgctgaggtagtgcccatggggcccccgccccctccaaagccgaaacagaccagagatagtactttcatggagaagttacatgcggtagatgaggagctggcttccagtccagtctgcatggattctttccagcccatggatgacagtctcattgcatttcgaacgcgttctaagatgcccctgaaagatgttcccctgggccaattagaggcagagctccaagctccagacatcactccagatatgtatgaccccaatacggcagatgatgaggactggaagatgtggctggggggacttatgaatgatgatgtggggaatgaagatgaagcagatgatgatgatgatccagaatataatttcctggaagacctcgatgaaccagacacagaggatttccggactgaccgggcagtgagaatcaccaaaaaggaagtaaatgagctgatggaagagctgtttgaaactttccaagatgagatgggattctccaacatggaagatgatggcccagaagaggaggagtgtgtagctgagcctcgtcctaactttaacacccctcaagctctacggtttgaggaaccactggccaacctgttaaatgaacaacatcggacagtgaaggagctatttgaacagctgaagatgaagaaatcttcagccaaacagctgcaggaagtagagaaggttaaaccccagagtgagaaagttcatcagactctgattctggacccagcacagaggaagagactccagcagcagatgcagcagcacgttcagctcttgacccaaatccaccttcttgccacctgcaaccccaacctcaatccggaggccactaccaccaggatatttcttaaagagctgggaacctttgctcaaagctccatcgcccttcaccatcagtacaaccccaagtttcagaccctgttccaaccctgtaacttgatgggagctatgcagctgattgaagacttcagcacacatgtcagcattgactgcagccctcataaaactgtcaagaagactgcgaatgaatttccctgtttgccaaagcaagtggcttggattctggccacaagcaaggttttcatgtatccagagttacttccagtgtgttccctgaaggcaaagaatccccaggataagatcgtcttcaccaaggctgaggacaatttgttagctttaggactgaagcattttgaaggaactgagtttcctaatcctctaatcagcaagtaccttctaacctgcaaaactgcccaccaactgacagtgagaatcaagaacctcaacatgaacagagctcctgacaacatcattaaattttataagaagaccaaacagctgccagtcctaggaaaatgctgtgaagagatccagccacatcagtggaagccacctatagagagagaagaacaccggctcccattctggttaaaggccagtctgccatccatccaggaagaactgcggcacatggctgatggtgctagagaggtaggaaatatgactggaaccactgagatcaactcagatcgaagcctagaaaaagacaatttggagttggggagtgaatctcggtacccactgctattgcctaagggtgtagtcctgaaactgaagccagttgccacccgtttccccaggaaggcttggagacagaagcgttcatcagtcctgaagcccctccttatccaacccagcccctctctccagcccagcttcaaccctgggaaaacaccagcccgatcaactcattcagaagcccctccgagcaaaatggtgctccggattcctcacccaatacagccagccactgttttacagacagttccaggtgtccctccactgggggtcagtggaggtgagagttttgagtctcctgcagcactgcctgctgtgccccctgaggccaggacaagcttccctctgtctgagtcccagactttgctctcttctgcccctgtgcccaaggtaatgctgccctcccttgccccttctaagtttcgaaagccatatgtgagacggagaccctcaaagagaagaggagtcaaggcctctccctgtatgaaacctgcccctgttatccaccaccctgcatctgttatcttcactgttcctgctaccactgtgaagattgtgagccttggcggtggctgtaacatgatccagcctgtcaatgcggctgtggcccagagtccccagactattcccatcactaccctcttggttaaccctacttccttcccctgtccattgaaccagtcccttgtggcctcctctgtctcacccttaattgtttctggcaattctgtgaatcttcctataccatccacccctgaagataaggcccacgtgaatgtggacattgcttgtgctgtggctgatggggaaaatgcctttcagggcctagaacccaaattagagccccaggaactatctcctctctctgctactgttttcccgaaagtggaacatagcccagggcctccactagcagatgcagagtgccaagaaggattgtcagagaatagtgcctgtcgctggaccgttgtgaaaacagaggaggggaggcaagctctggagccgctccctcagggcatccaggagtctctaaacaaccctacccctggggatttagaggaaattgtcaagatggaacctgaagaagctagagaggaaatcagtggatcccctgagcgtgatatttgtgatgacatcaaagtggaacatgctgtggaattggacactggtgccccaagcgaggagttgagcagtgctggagaagtaacgaaacagacagtcttacagaaggaagaggagaggagtcagccaactaaaaccccttcatcttctcaagagccccctgatgaaggaacctcagggacagatgtgaacaaaggatcatcaaagaatgctttgtcctcaatggatcctgaagtgaggcttagtagccccccagggaagccagaagattcatccagtgttgatggtcagtcagtggggactccagttgggccagaaactggaggagagaagaatgggccagaagaagaggaagaagaggactttgatgacctcacccaagatgaggaagatgaaatgtcatcagcttctgaggaatctgtgctttctgtcccagaactccaggtgagagctggagaatattctcaagtatttcgtggactcagtaatatgtatcacttattgatatgccacctgcttgcttgctgcactatggatagtcctaaaatcatttgtattctcgagggtaagcctatccctaaccctctcctcggtctcgattctacgtaatga

SEQ ID NO: 10 - DNA, 1068 bp, Homo sapiens
  source 1..1068: mol_type = genomic DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgat

SEQ ID NO: 11 - DNA, 1122 bp, Homo sapiens
  source 1..1122: mol_type = genomic DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgatctcgagggtaagcctatccctaaccctctcctcggtctcgattctacgtaatga

SEQ ID NO: 12 - DNA, 891 bp, Homo sapiens
  source 1..891: mol_type = genomic DNA
atgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttg

SEQ ID NO: 13 - DNA, 945 bp, Homo sapiens
  source 1..945: mol_type = genomic DNA
atgatgtcttatcttaagcaaccgccttacgcagtcaatgggctgagtctgaccacttcgggtatggacttgctgcacccctccgtgggctacccggggccctgggcttcttgtcccgcagccaccccccggaaacagcgccgggagaggacgacgttcactcgggcgcagctagatgtgctggaagcactgtttgccaagacccggtacccagacatcttcatgcgagaggaggtggcactgaaaatcaacttgcccgagtcgagggtgcaggtatggtttaagaatcgaagagctaagtgccgccaacaacagcaacaacagcagaatggaggtcaaaacaaagtgagacctgccaaaaagaagacatctccagctcgggaagtgagttcagagagtggaacaagtggccaattcactcccccctctagcacctcagtcccgaccattgccagcagcagtgctcctgtgtctatctggagcccagcttccatctccccactgtcagatcccttgtccacctcctcttcctgcatgcagaggtcctatcccatgacctatactcaggcttcaggttatagtcaaggatatgctggctcaacttcctactttgggggcatggactgtggatcatatttgacccctatgcatcaccagcttcccggaccaggggccacactcagtcccatgggtaccaatgcagtcaccagccatctcaatcagtccccagcttctctttccacccagggatatggagcttcaagcttgggttttaactcaaccactgattgcttggattataaggaccaaactgcctcctggaagcttaacttcaatgctgactgcttggattataaagatcagacatcctcgtggaaattccaggttttgctcgagggtaagcctatccctaaccctctcctcggtctcgattctacgtaatga